(19) United States
(12) Patent Application Publication
(10) Pub. No.: US 2018/0259531 A1
(43) Pub. Date: Sep. 13, 2018

(54) MEASUREMENT OF MOLECULAR FLUX RATES BY QUANTIFYING ISOTOPOLOGUE ABUNDANCES USING HIGH RESOLUTION MASS SPECTROMETRY

(71) Applicant: KINEMED, INC., Piedmont, CA (US)

(72) Inventors: Marc K. HELLERSTEIN, Kensington, CA (US); Thomas E. ANGEL, Trappe, PA (US)

(73) Assignee: KineMed, Inc., Piedmont, CA (US)

(21) Appl. No.: 15/758,261

(22) PCT Filed: Sep. 7, 2016
(86) PCT No.: PCT/US2016/050578

§ 371 Date: Mar. 7, 2018

Related U.S. Application Data
(60) Provisional application No. 62/215,643, filed on Sep. 8, 2015.

(51) Int. Cl. G01N 33/58; G01N 33/68

(52) U.S. Cl. CPC G01N 33/58; G01N 2458/15; G01N 33/6848

(57) ABSTRACT

Provided herein are methods for measuring a molecular flux rate based on analysis of isotopologue abundance within a mass isotopomer, e.g., using a high resolution mass spectrometric measurement. Such methods may be used, inter alia, to calculate a fraction of newly synthesized target molecules of interest, a replacement rate of target molecules of interest, and/or a rate of breakdown or degradation of target molecules of interest, e.g., based on isotopologue relative abundance.

### FIELD

The present disclosure relates generally to methods for measuring a molecular flux rate based on analysis of isotopologue abundance within a mass isotopomer, e.g., using a high resolution mass spectrometric measurement.

### BACKGROUND

Measurements of molecular flux rates, or kinetics, of processes that are causal in disease represent the next generation of medical biomarkers (Hellerstein, M. K. (2003) Annu. Rev. Nutr. 23:379-402; Turner, S. M. and Hellerstein, M. K. (2005) Curr. Opin. Drug Discov. Devel. 8:115-126; Hellerstein, M. K. (2008) Metab. Eng. 10:1-9; and Hellerstein, M. K. (2008) J. Pharmacol. Exp. Ther. 325:1-9). More efficient drug development, personalized medicine, medical diagnostics and wellness monitoring would all be considerably advanced by the availability of objective metrics that revealed the rates at which pathogenic molecular processes are occurring in living humans. Measurement of molecular kinetics or flux rates in vivo generally requires perturbing a system by introducing a tag, or isotopic label, and measuring its rate of flow into molecules of interest. The reason for this is that introduction of a label results in an asymmetry in the dimension of time (i.e., label was not present, then it was), so that time-dependent changes, or kinetic transients, can be measured, and the rates of observable processes can be calculated.

Over the past three decades, kinetic measurements in living systems have evolved from predominantly using radioactive isotopic tracers to the use of non-radioactive, stable isotopic tracers, particularly heavy water (²H₂O). The latter are generally measured by use of mass spectrometry (MS) or nuclear magnetic resonance. This transition was initially largely motivated by the safety and toxicity problems associated with and caused by radioisotopes, which are avoided by use of stable isotopes (Wolfe, R. R. and Chinkes, D. L. (2004) Isotope Tracers in Metabolic Research: Principles and Practice of Kinetic Analysis, 2^{nd} ed. Wiley). As mass spectrometers have dramatically improved, however, it has become apparent that stable isotope-MS methods provide a number of unique capabilities for molecular kinetic measurements that are not possible with radioisotope approaches (see, e.g., Hellerstein, M. K. and Neese, R. A. (1992) Am. J. Physiol. 263:E988-1001; and Hellerstein, M. K. and Neese, R. A. (1999) Am. J. Physiol. 276:E1146-1170). Two of the basic advantages provided by stable isotope-MS approaches are the capacity to measure different patterns of isotope labeling within individual molecules and the ability to detect and analyze molecular fragment ions.

Radioisotopic measurements—e.g., through liquid scintillation counting or Geiger counting—measure the total amount of label in a population of molecules, with the result typically expressed as disintegrations per minute (dpm)/mole (Wolfe, R. R. and Chinkes, D. L. (2004) Isotope Tracers in Metabolic Research: Principles and Practice of Kinetic Analysis, 2^{nd} ed. Wiley). The pattern or variability of labeling among individual molecules in the population, however, cannot be measured by this approach because individual molecules are not isolated, identified or detected.

In contrast, MS analysis is capable of detecting the relative abundances of different molecular species, or isotopic isomers, that are present in a sample. One category of isotopic isomers is mass isotopomers, which are defined as isotopic isomers that differ in elemental isotope composition and nominal mass—e.g., single-labeled vs. double-labeled species of a molecule. Many applications of mass isotopomer analysis have been discovered (Hellerstein, M. K. and Neese, R. A. (1992) Am. J. Physiol. 263:E988-1001; Hellerstein, M. K. and Neese, R. A. (1999) Am. J. Physiol. 276:E1146-1170; Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286:E577-588), in particular through use of “mass isotopomer distribution analysis” (MIDA; see, e.g., U.S. Pat. No. 5,338,686). MIDA is a method that includes quantifying changes in the relative abundances of different mass isotopomers of a molecule (e.g., the relative proportions of unlabeled, single-labeled, double-labeled, etc. molecular species), compared to natural abundances of the mass isotopomers, and then inferring from this internal labeling pattern the isotopic content of the true biosynthetic precursor pool at the intracellular site of biosynthesis, through application of equations based on the binomial or multinomial distribution. The pattern of excess abundances or enrichments of each mass, termed (EM1, EM2, EM3, etc.) thereby reveals the fraction of labeled atoms in the biosynthetic precursor pool (Hellerstein, M. K. and Neese, R. A. (1992) Am. J. Physiol. 263:E988-1001; and Hellerstein, M. K. and Neese, R. A. (1999) Am. J. Physiol. 276:E1146-1170). Knowledge of the label enrichment of the true biosynthetic precursor pool, e.g., calculated in this manner, allows essentially all parameters related to molecular fluxes of the molecule to be calculated with rigorous accuracy. MIDA has been called “the equation for biosynthesis.” (Hellerstein, M. K. and Neese, R. A. (1992) Am. J. Physiol. 263:E988-1001; and Hellerstein, M. K. and Neese, R. A. (1999) Am. J. Physiol. 276:E1146-1170). The capacity to measure mass isotopomer abundances (MIAs) and mass isotopomer distributions (MIDs) has been applied to kinetic analysis of lipids (Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286:E577-588), intermediary metabolites (Neese, R. A. et al. (1995) J. Biol. Chem. 270:14452-14466; Hellerstein, M. K. et al. (1997) Am. J. Physiol. 272:E163-172; Hellerstein, M. K. et al. (1997) J. Clin. Invest. 100:1305-1309; and Louie, K. B. et al. (2013) Sci. Rep. 3:1656), proteins (Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814) and cells (Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057). Of note, all of these molecular flux rates can be measured by use of ²H₂O labeling.

Broad application of molecular kinetic measurements for routine clinical medical management and drug development would have a potentially transformative effect on these areas of biomedicine. The power of kinetic measurements to capture the activity of disease-driving processes could radically advance personalized medicine and the efficiency of testing drug candidates in FDA trials (Messmer, B. T. et al. (2005) J. Clin. Invest. 115:755-764; and Decaris, M. L. et al. (2015) PLoS One 10:e0123311).

There are major technical limitations and constraints, however that continue to prevent broad or routine applications of mass spectrometric kinetic analysis in clinical medicine and drug development. The most important of these limitations is the inability of the current generation of mass spectrometers to achieve sufficiently accurate and precise measurements of MIAs and MIDs in organic biomolecules for routine medical applications. Mass spectrometers including quadrupole, ion trap, time-of-flight (ToF), magnetic sector, and Fourier transform-ion cyclotron resonance (FT-ICR) mass spectrometers (including Orbitrap mass analyzers), to name a few, along with hybrid instrument modalities, have not had sufficient analytic precision and accuracy for MIAs and MIDs to allow routine application of molecular kinetic methods in clinical medicine or in drug development settings, particularly with the relatively low isotopic perturbations induced by ²H₂O labeling in the clinical setting, where dose and duration of ²H₂O administration are limited by safety and practical factors.

Accordingly, methods that take advantage of the high mass resolution, mass measurement accuracy, and sensitivity of high resolution mass spectrometers; provide improved accuracy and precision for MIA and MID measurements as compared to current instruments; and are less susceptible to interferences from contaminating ions in complex analytic matrices would be useful and broadly applicable across biology, medicine, and clinical practice.

All publications, patents, and patent applications cited herein are hereby incorporated by reference in their entirety for all purposes.

### BRIEF SUMMARY

In the field of measuring molecular flux rates, a central analytic requirement is accurate and precise measurements of changes in relative abundances of potentially labeled species of molecules of interest after introduction of a labeled metabolic precursor. Analytic imprecision and inaccuracy of current measurement techniques for quantifying relative abundancies of different labeled species (specifically, mass isotopomers) in targeted molecules is a major problem currently holding back broad application of molecular kinetics (the measurement of molecular flux rates) in medical practice and biomedical research, particularly for use of the most universal and clinically useful labeling approach (labeling with heavy water [²H₂O]), which generates relatively modest changes in mass isotopomer abundances.

As described herein, measuring the relative abundances of isotopologues within mass isotopomers of a molecule, which differ in mass by millidaltons, by use of high mass-resolution mass spectrometers (e.g., FT-ICR instruments) provides analytic advantages, particularly for ²H₂O labeling approaches. There are numerous advantages and potential biomedical applications of isotopologue analysis in mass isotopomers with high resolution mass spectrometers for measuring molecular flux rates in clinical medicine and biomedical research, particularly when changes in mass isotopomer abundances are relatively modest, as occurs for low dose or brief duration ²H₂O labeling protocols that are optimal for broad medical diagnostic applications of molecular flux rate measurements. Accordingly, provided herein, inter alia, are methods for metabolically labeling and analyzing relative abundances of isotopologues in selected mass isotopomers by use of high mass-resolution mass spectrometers, as well as methods for calculating molecular flux rates of molecules by this approach.

Certain aspects of the present disclosure relate to methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and (g) calculating a fraction of newly synthesized target molecules of interest based on the comparison of the relative abundance of the first isotopologue and the control relative abundance of the first isotopologue. In some embodiments, the methods further comprise calculating a replacement rate of the target molecules of interest based on the calculated fraction of newly synthesized target molecules of interest. In some embodiments, the methods further comprise obtaining from the subject at least a second biological sample comprising the one or more stable isotope-labeled target molecules of interest, wherein the first and second biological samples are obtained at different times, and wherein calculating the fraction of newly synthesized target molecules of interest comprises calculating a fraction of target molecules of interest synthesized before obtaining the first biological sample and a fraction of target molecules of interest synthesized before obtaining the second biological sample.

Other aspects of the present disclosure relate to methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and (g) calculating a rate of breakdown or degradation of the target molecules of interest based on the comparison of the relative abundance of the first isotopologue and the control relative abundance of the first isotopologue. In some embodiments, the methods further comprise calculating a replacement rate of the target molecules of interest based on the calculated rate of breakdown or degradation of the target molecules of interest.

In some embodiments of any of the above embodiments, the stable isotope-labeled precursor molecule is ²H₂O. In certain embodiments, the first isotopologue is a ²H-isotopologue, and the second isotopologue is a ¹³C-isotopologue. In some embodiments of any of the above embodiments, the stable isotope-labeled precursor molecule is selected from the group consisting of a ¹⁵N-labeled amino acid, a ¹⁵N-labeled polypeptide, and a ¹⁵N-labeled inorganic nitrogenous compound. In some embodiments of any of the above embodiments, the stable isotope-labeled precursor molecule is selected from the group consisting of a ¹³C-labeled amino acid, a ¹³C-labeled polypeptide, a ¹³C-labeled organic metabolite, and a ¹³C-labeled inorganic carbon compound. In some embodiments of any of the above embodiments, the stable isotope-labeled precursor molecule is ¹⁷O-labeled H2O or ¹⁸O-labeled H₂O.

In some embodiments of any of the above embodiments, the mass isotopomer is an M1-mass isotopomer.

Other aspects of the present disclosure relate to methods for measuring a molecular flux rate based on analysis of isotopologue abundance within a mass isotopomer, comprising: (a) administering deuterated water (²H₂O) to a subject for a period of time sufficient for the deuterium of the ²H₂O to enter into a biosynthetic precursor pool and label one or more target molecules of interest to produce one or more deuterated target molecules of interest; (b) obtaining from the subject a biological sample comprising the one or more deuterated target molecules of interest; (c) enriching or isolating the one or more deuterated target molecules of interest from said biological sample; (d) performing a high resolution mass spectrometric measurement of an abundance of a first isotopologue and an abundance of a second isotopologue from said enriched or isolated one or more deuterated target molecules of interest, wherein the first and the second isotopologues have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the ²H₂O; and (g) calculating a fraction of newly synthesized target molecules of interest based on the comparison of the relative abundance of the first isotopologue and the control relative abundance of the first isotopologue. In some embodiments, the methods further comprise calculating a replacement rate of the target molecules of interest based on the calculated fraction of newly synthesized target molecules of interest. In some embodiments, the methods further comprise obtaining from the subject at least a second biological sample comprising the one or more stable isotope-labeled target molecules of interest, wherein the first and second biological samples are obtained at different times, and wherein calculating the fraction of newly synthesized target molecules of interest comprises calculating a fraction of target molecules of interest synthesized before obtaining the first biological sample and a fraction of target molecules of interest synthesized before obtaining the second biological sample.

Other aspects of the present disclosure relate to methods for measuring a molecular flux rate based on analysis of isotopologue abundance within a mass isotopomer, comprising: (a) administering deuterated water (²H₂O) to a subject for a period of time sufficient for the deuterium of the ²H₂O to enter into a biosynthetic precursor pool and label one or more target molecules of interest to produce one or more deuterated target molecules of interest; (b) obtaining from the subject a biological sample comprising the one or more deuterated target molecules of interest; (c) enriching or isolating the one or more deuterated target molecules of interest from said biological sample; (d) performing a high resolution mass spectrometric measurement of an abundance of a first isotopologue and an abundance of a second isotopologue from said enriched or isolated one or more deuterated target molecules of interest, wherein the first and the second isotopologues have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the ²H₂O; and (g) calculating a rate of breakdown or degradation of the target molecules of interest based on the comparison of the relative abundance of the first isotopologue and the control relative abundance of the first isotopologue. In some embodiments, the methods further comprise calculating a replacement rate of the target molecules of interest based on the calculated rate of breakdown or degradation of the target molecules of interest.

In some embodiments of any of the above embodiments, the one or more target molecules of interest are peptides or amino acids isolated from one or more polypeptides. In some embodiments of any of the above embodiments, the one or more target molecules of interest are deoxyribose molecules isolated from DNA. In some embodiments, the first and the second isotopologues on which the high resolution mass spectrometric measurement is performed are derived from a fragment ion, the fragment ion being derived from the one or more stable isotope-labeled or deuterated target molecules of interest. In some embodiments, the one or more target molecules of interest are isolated from a cell.

In some embodiments of any of the above embodiments, the high resolution mass spectrometric measurement is performed using a high resolution mass spectrometer capable of quantifying isotopologues that differ in mass by nine or fewer millidaltons. In some embodiments, the high resolution mass spectrometric measurement is performed using a high resolution mass spectrometer capable of quantifying isotopologues that differ in mass by three or fewer millidaltons. In some embodiments of any of the above embodiments, the abundances of the first isotopologue and the second isotopologue are of comparable peak heights or signal intensities. In some embodiments of any of the above embodiments, calculating the fraction of newly synthesized target molecules of interest, the replacement rate of the target molecules of interest, the rate of breakdown or degradation of the target molecules of interest, or any combination thereof is used as a diagnostic test. In certain embodiments, the diagnostic test is used in the diagnosis, management, or treatment selection of a human or veterinary patient. In some embodiments of any of the above embodiments, the subject is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DESCRIPTION OF THE FIGURES

FIG. 1 shows a plateaued ²H₂O body water enrichment of approximately 0.75% (p) and an average or effective p of 0.5% resulting from a standard low-dose ²H₂O-labeling protocol.

FIG. 2 shows a theoretical fractional synthesis rate of plasma haptoglobin in a human subject over time obtained from a standard low-dose ²H₂O-labeling protocol.

FIG. 3 shows relative abundances of the ¹³C and the ²H isotopologues of the M1-mass isotopomer in a fragment ion of serine from a haptoglobin peptide obtained after a standard low-dose ²H₂O-labeling protocol. In a sample from a subject with an average body water ²H₂O exposure of 0.50% over a period of 12 hours (see FIG. 1), the fraction of newly synthesized haptoglobin molecules will be ˜15% (see FIG. 2). This will result in a change in the ²H-isotopologue % relative abundance from a very low natural abundance value (lowest ²H-trace labeled with triangles) to ˜20% relative abundance (middle ²H-trace labeled with squares), as compared to the ¹³C-isotopologue of the M1-mass isotopomer. The values shown are from the ratio of the abundance of the ²H-isotopologue of the M1-mass isotopomer to the sum of (i) the abundance of the ²H-isotopologue of the M1-mass isotopomer and (ii) the abundance of the ¹³C-isotopologue of the M1-mass isotopomer (expressed algebraically ²H/(²H+¹³C)). The theoretical maximal % relative abundance for the ²H-isotopologue of the M1-mass isotopomer under these labeling conditions, representing the value for this fragment ion of serine from this peptide in newly synthesized molecules of haptoblobin, is ˜60% of the abundance of the ¹³C-isotopologue (highest ²H trace in diamonds). This measurable ˜20% relative abundance of the ²H-isotopologue compared to the ¹³C-isotopologue of the M1-mass isotopomer after 12 hours of label exposure is a much higher signal than the <1% change in relative abundance of the M1-mass isotopomer that is observable under these conditions (not shown).

FIGS. 4A & 4B illustrate modeled mass isotopomer (FIG. 4A) and isotopologue (FIG. 4B) spectra for a sample peptide. In both graphs, plots of abundance vs. mass are shown.

FIG. 5 shows modeled isotopomer and isotopologue abundance profiles for a sample peptide labeled under conditions where body water was at 1.0% enrichment with ²H₂O. Isotopologue abundance profiles for the M1 and M2 isotopomers are shown. Large changes in relative abundance signal between unlabeled and labeled species are observed for the ²H-isotopologues of the M1 and M2 mass isotopomers.

FIGS. 6A-6C show mass isotopomer distribution of unlabeled deoxyribose (FIG. 6A), distribution of isotopologues in the M1 mass isotopomer of unlabeled deoxyribose (FIG. 6B), and distribution of isotopologues in the M1 mass isotopomer of heavy water (²H₂O) labeled deoxyribose for a body water ²H enrichment of 2% (FIG. 6C). The change in peak theoretical label % relative abundance of the ²H-isotopologue compared to the ¹³C-isotopologue of the M1-mass isotopomer, as calculated from the ratio of the abundance of the ²H-isotopologue of the M1-mass isotopomer to the sum of (i) the abundance of the ²H-isotopologue of the M1-mass isotopomer and (ii) the abundance of the ¹³C-isotopologue of the M1-mass isotopomer (expressed algebraically as ²H/(²H+¹³C)), is very large, as is apparent from comparing FIG. 6B to FIG. 6C.

FIG. 7 shows a modeled graph of the ratio of relative abundances of ²H and ¹³C-isotopologues in the M1-mass isotopomer in deoxyribose, calculated as the ratio of the abundance of the ²H-isotopologue of the M1-mass isotopomer to the sum of (i) the abundance of the ²H-isotopologue of the M1-mass isotopomer and (ii) the abundance of the ¹³C-isotopologue of the M1-mass isotopomer (expressed algebraically ²H/(²H+¹³C)), versus the fractional synthesis (f) at different body water ²H₂O exposures (p). Even at relatively low values off, such as 20%, the change in ratio of relative abundances of ²H and ¹³C-isotopologues in the M1-mass isotopomer in deoxyribose, is substantial, resulting in a large analytic signal for measurement off.

FIG. 8A shows measurements of relative abundances of the ²H and ¹³C-isotopologues in the M1-mass isotopomer of deoxyribose from mixtures of DNA isolated from heavy water-labeled and unlabeled cells, measured by FT-ICR mass spectrometry, as compared to measurements of mass isotopomer abundances by gas chromatography/mass spectrometry (GC/MS), from the same analytic samples. FIG. 8A shows the FT-ICR analytic traces of the peaks for the ²H and ¹³C-isotopologues in the M1-mass isotopomer of the sodiated species of deoxyribose, from progressively increasing mixtures of labeled DNA and unlabeled DNA, wherein labeled DNA was added to represent from 0 to 40% of the mixture of total DNA present. Labeled DNA was isolated from bone marrow cells of rodents after several weeks' exposure to heavy water (²H₂O) in drinking water, to allow essentially complete replacement of bone marrow cells by newly divided cells. Unlabeled DNA was from liver cells of rodents with no exposure to heavy water.

FIG. 8B shows a graph of the relationship between the measured relative abundances of the ²H and ¹³C-isotopologues in the M1-mass isotopomer (based on the measured peak heights shown in Table 2) and the equation relating these measured relative abundances of isotopologues [²H/²H+¹³C)] to theoretical or calculated relative abundances of the ²H and ¹³C-isotopologues in the M1-mass isotopomer, from which the fraction of newly synthesized deoxyribose molecules can be calculated.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Described herein are methods for measuring a molecular flux rate based on analysis of isotopologue abundance within a mass isotopomer, e.g., using a high resolution mass spectrometric measurement. These methods are based at least in part on the discovery described herein that metabolically labeling and analyzing relative abundances of isotopologues in selected mass isotopomers (e.g., through use of high mass-resolution mass spectrometry) may allow for the calculation of molecular flux rates with greater accuracy and/or precision than standard techniques. This is thought to be particularly advantageous for applications in which observed changes in mass isotopomer abundances are relatively modest, as with ²H₂O labeling protocols that are optimal for medical diagnosis and testing.

In certain aspects, provided herein are methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and (g) calculating a fraction of newly synthesized target molecules of interest based on the comparison of the relative abundance of the first isotopologue and the control relative abundance of the first isotopologue.

In other aspects, provided herein are methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and (g) calculating a rate of breakdown or degradation of the target molecules of interest based on the comparison of the relative abundance of the first isotopologue and the control relative abundance of the first isotopologue.

In yet other aspects, provided herein are methods for measuring a molecular flux rate based on analysis of isotopologue abundance within a mass isotopomer, comprising: (a) administering deuterated water (²H₂O) to a subject for a period of time sufficient for the deuterium of the ²H₂O to enter into a biosynthetic precursor pool and label one or more target molecules of interest to produce one or more deuterated target molecules of interest; (b) obtaining from the subject a biological sample comprising the one or more deuterated target molecules of interest; (c) enriching or isolating the one or more deuterated target molecules of interest from said biological sample; (d) performing a high resolution mass spectrometric measurement of an abundance of a first isotopologue and an abundance of a second isotopologue from said enriched or isolated one or more deuterated target molecules of interest, wherein the first and the second isotopologues have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the ²H₂O; and (g) calculating a fraction of newly synthesized target molecules of interest based on the comparison of the relative abundance of the first isotopologue and the control relative abundance of the first isotopologue.

In still other aspects, provided herein are methods for measuring a molecular flux rate based on analysis of isotopologue abundance within a mass isotopomer, comprising: (a) administering deuterated water (²H₂O) to a subject for a period of time sufficient for the deuterium of the ²H₂O to enter into a biosynthetic precursor pool and label one or more target molecules of interest to produce one or more deuterated target molecules of interest; (b) obtaining from the subject a biological sample comprising the one or more deuterated target molecules of interest; (c) enriching or isolating the one or more deuterated target molecules of interest from said biological sample; (d) performing a high resolution mass spectrometric measurement of an abundance of a first isotopologue and an abundance of a second isotopologue from said enriched or isolated one or more deuterated target molecules of interest, wherein the first and the second isotopologues have different masses; (e) calculating a relative abundance of the first isotopologue based on the abundance of the first isotopologue and the abundance of the second isotopologue; (f) comparing the relative abundance of the first isotopologue to a control relative abundance of the first isotopologue, wherein the control relative abundance is the relative abundance of the first isotopologue from the one or more target molecules of interest before or without administration of the ²H20; and (g) calculating a rate of breakdown or degradation of the target molecules of interest based on the comparison of the relative abundance of the first isotopologue and the control relative abundance of the first isotopologue.

### I. General Techniques

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) 3. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

U.S. Pat. No. 8,129,335 provides methods and disclosures that may be useful for practice of methods described herein.

### II. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Kinetic parameters” and “molecular flux rates” may be used interchangeably herein and may refer to the rate of synthesis, breakdown, and/or transport of a macromolecule (e.g., a polypeptide or polynucleotide). “Kinetic parameters” or “molecular flux rates” also refer to a macromolecule's input into or removal from a pool of macromolecules, and are therefore synonymous with the flow into and out of the pool of macromolecules.

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Isotopologues” refer to isotopic homologues or molecular species that have identical elemental compositions but differ in isotopic content (e.g., CH₃NH₂ vs. CH₃NHD in the example above). Isotopologues are defined by their isotopic composition; therefore, each isotopologue has a unique exact mass but may not have a unique structure. An isotopologue usually includes of a family of isotopic isomers (isotopomers) which differ by the location of the isotopes on the molecule (e.g., CH₃NHD and CH₂DNH₂ are the same isotopologue but are different isotopomers). For example, ethanol, with a molecular formula of ¹²C₂ ¹H₆ ¹⁶O₁ for the monoisotopic M0 isotopomer, has 3 possible M1 isotopologues contributing to the M1-mass isotopomer (which is nominally 1 dalton heavier than the M0 mass isotopomer); these have isotopic elemental chemical formulas of ¹³C₁ ¹²C₁ ¹H₆ ¹⁶O₁, ¹²C₂ ¹H₆ ¹⁷O¹, and ¹²C₂ ²H₁ ¹H₅ ¹⁶O₁.

“Mass isotopomer” refers to family of isotopic isomers that is grouped on the basis of nominal mass, rather than isotopic composition or exact mass. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). For example, the isotopologues CH₃NHD, ¹³CH₃NH₂, and CH₃ ¹⁵NH₂ are all of the same nominal mass, and hence are the same mass isotopomers, even though each may have a different exact mass. Each mass isotopomer is therefore typically composed of more than one isotopologue and has more than one exact mass. The mass isotopomer lowest in mass is represented as M0; for most organic molecules, this is the species containing all ¹²C, ¹H, ¹⁶O, ¹⁴N, etc. Other mass isotopomers are distinguished by their mass differences from M0 (M1, M2, etc.). For a given mass isotopomer, the location or position of isotopes within the molecule is not specified and may vary (i.e., “positional isotopomers” are not distinguished).

“Mass isotopomer envelope” refers to the set of mass isotopomers associated with a molecule or ion fragment.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonymously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, CI, Br, and I, the isotopic composition of the isotopologue with the lowest mass is unique and unambiguous because the most abundant isotopes of these elements are also the lowest in mass. The monoisotopic mass is abbreviated as m.sub.0, and the masses of other mass isotopomers are identified by their mass differences from m.sub.0 (m₁, m₂, etc.).

A “high resolution mass spectrometric measurement” and terms related thereto may refer to a measurement that is capable of distinguishing isotopologues within the same mass isotopomer using mass spectrometry. For example, a high resolution mass spectrometric measurement may distinguish one or more mass differences among ¹⁵N, ¹³C, ¹⁷O, and/or ²H-labeled isotopologues in the same mass isotopomer. To use the M1-mass isotopomer of ethanol as a specific example, a high resolution mass spectrometric measurement may resolve one or more isotopologues having isotopic elemental chemical formulas of ¹³O₁ ¹²C₁ ¹H₆ ¹⁶O₁, ¹²C₂ ¹H₆ ¹⁷O¹, and ¹²C₂ ²H₁ ¹H₅ ¹⁶O₁.

“Isotope-labeled water” includes water labeled with one or more specific heavy isotopes of either hydrogen or oxygen. Specific examples of isotope-labeled water include ²H₂O, ³H₂O, and H₂¹⁸O.

“Stable isotope-labeled precursor molecules” refer to a metabolite precursor that contains a stable isotope of an element that differs from the most abundant isotope of the element present in nature or cells, tissues, or organisms. Stable isotopic labels include specific heavy isotopes of elements, present in biomolecules, such as ²H, ¹³C, ¹⁵N, ¹⁷O, and ¹⁸O. Isotope labeled organic metabolite precursors include but are not limited to ²H₂O, ¹⁵NH₃, ¹³CO₂, H¹³CO₃, H₂¹⁷O, H₂¹⁸O, ²H-labeled amino acids, ¹³C-labeled amino acids, ¹³C-labeled polypeptides, ¹³C-labeled organic metabolites, ¹³C-labeled inorganic carbon compounds, ¹⁵N-labeled amino acids, ¹⁵N-labeled polypeptides, ¹⁵N-labeled inorganic nitrogenous compounds, ¹⁷O-labeled amino acids, ¹⁸O-labeled amino acids.

“Enriching” refers to methods of removing one or more components of a mixture of other similar compounds. As used herein, the term “partially purifying” may be used interchangeably.

“Isolating” refers to separating one compound from a mixture of compounds. For example, “isolating a macromolecule” refers to separating one specific macromolecule from all other macromolecules in a mixture of one or more macromolecules.

“Relative abundance” refers to a comparison (e.g., a ratio or other mathematical operation) between an abundance (e.g., quantitative abundance), peak height, peak area or other parameter known in the art for an amount of a first molecular species (e.g., an isotopologue) and an abundance (e.g., quantitative abundance), peak height, peak area or other parameter known in the art for an amount of at least a second other molecular species (e.g., one or more other isotopologues), as measured by a mass spectrometer. As used herein, a relative abundance may be expressed by any of several mathematical equations, including but not limited to a ratio of the abundance of the first isotopologue to a sum of (i) the abundance of the first isotopologue and (ii) an abundance of a second isotopologue; a ratio of abundance of a first isotopologue to the abundance of a second isotopologue; a ratio of abundance of a first isotopologue to a sum of (i) the abundance of the first isotopologue and (ii) an abundance of a second and other isotopologues.

A “biological sample” encompasses any sample obtained from a cell, tissue, or organism. The definition encompasses blood and other liquid samples of biological origin, that are accessible from an organism through sampling by minimally invasive or non-invasive approaches (e.g., urine collection, blood drawing, needle aspiration, and other procedures involving minimal risk, discomfort or effort), as well as portions of solid tissue that may be obtained, e.g., using a surgical biopsy; surgical removal; percutaneous, endoscopic, transvascular, radiographic-guided or other non-surgical biopsy; euthanizing an experimental animal and removing tissue; collecting ex vivo experimental preparations; removing tissue at post-mortem examination; or other methods of collecting tissues. The definition also includes samples that have been manipulated in any way after their procurement, such as by treatment with reagents, solubilization, or enrichment for certain components, such as proteins or organic metabolites. The term “biological sample” also encompasses a clinical sample such as serum, plasma, other biological fluid, or tissue samples, and also includes cells in culture, cell supernatants and cell lysates. A volume of a body fluid may be used to refer to a liquid biological sample.

“Isotopically perturbed” refers to the state of an element or molecule that results from the explicit incorporation of an element or molecule with a distribution of isotopes that differs from the distribution found in nature, whether a naturally less abundant isotope is present in excess (enriched) or in deficit (depleted).

“Monomer” refers to a chemical unit that combines during the synthesis of a polymer and which is present two or more times in the polymer.

“Polymer” refers to a molecule synthesized from and containing two or more repeats of a monomer.

“Protein” (the term “polypeptide” may be used interchangeably herein) refers to a polymer of amino acids. As used herein, a “protein” may refer to long amino acid polymers as well as short polymers such as peptides.

“Polynucleotide” refers to a polymer of nucleotides. As used herein, a “polynucleotide” may refer to polymers such as DNA, RNA, cDNA, and so forth, and is meant to include polymers of naturally occurring polynucleotides and polymers of modified or unnatural polynucleotides, as well as polymers having a mixture or combination thereof.

### III. Methods of the Disclosure

The present disclosure is directed to methods measuring one or more molecular flux rate(s) based on analysis of isotopologue abundance within a mass isotopomer. The methods may include administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; obtaining from the subject a biological sample comprising the one or more stable isotope-labeled target molecules of interest; enriching or isolating the one or more stable isotope-labeled target molecules of interest from said biological sample; performing a high resolution mass spectrometric measurement of a relative abundance of a first isotopologue from said enriched or isolated one or more stable isotope-labeled target molecules of interest, wherein the relative abundance of the first isotopologue is a ratio of abundance of the first isotopologue to a sum of (i) the abundance of the first isotopologue and (ii) an abundance of a second isotopologue from said enriched or isolated one or more stable isotope-labeled target molecules of interest, wherein the first and the second isotopologues are part of the same mass isotopomer and have different masses; and (e) comparing the relative abundance of the first isotopologue to a control relative abundance of the first isotopologue, wherein the control relative abundance is a ratio of abundance of the first isotopologue from the one or more target molecules of interest before or without administration of the stable isotope-labeled precursor molecule to a sum of (i) the abundance of the first isotopologue before or without administration of the stable isotope-labeled precursor molecule and (ii) an abundance of the second isotopologue from the one or more target molecules of interest without administration of the stable isotope-labeled precursor molecule.

In some embodiments, comparing the relative abundance of the first isotopologue to the control relative abundance of the first isotopologue may be used to calculate a fraction of newly synthesized target molecules of interest, e.g., based on the change in relative abundance of the first and the second isotopologues in the enriched or isolated stable isotope-labeled target molecules of interest collected during or after administering the stable isotope-labeled precursor molecule to the subject, as compared to the relative abundance of the first and the second isotopologues in the target molecules of interest before or without administering the stable isotope-labeled precursor molecule. In some embodiments, this fraction of newly synthesized target molecules of interest may subsequently be used, inter alia, to calculate a replacement rate of the target molecules of interest.

In other embodiments, comparing the relative abundance of the first isotopologue to the control relative abundance of the first isotopologue may be used to calculate a rate of breakdown or degradation of the target molecules of interest, e.g., based on the rate of decrease over time in the relative abundance of the first and the second isotopologues in the enriched or isolated stable isotope-labeled target molecules of interest in the enriched or isolated stable isotope-labeled target molecules of interest collected during the period after administering the stable isotope-labeled precursor molecule to the subject.

A. Administering a Stable Isotope-Labeled Precursor Molecule

1. Labeled Precursor Molecules

a. Isotope Labels

The first step in measuring molecular flux rates involves administering an isotope-labeled precursor molecule to a cell, tissue, or organism. In some embodiments, the isotope labeled precursor molecule may be a stable isotope. Isotope labels that can be used include, but are not limited to, ²H, ¹³C, ¹⁵N, ¹⁷O, ¹⁸O, or other stable isotopes of elements present in organic systems. In one embodiment, the isotope label is ²H.

b. Precursor Molecules

The precursor molecule may be any molecule having an isotope label that is incorporated into an organic metabolite, such as a polypeptide or polynucleotide. Isotope labels may be used to modify all precursor molecules disclosed herein to form isotope-labeled precursor molecules.

In some embodiments, the entire precursor molecule may be incorporated into one or more organic metabolites. Alternatively, a portion of the precursor molecule may be incorporated into one or more organic metabolites.

Precursor molecules may include, but are not limited to, CO₂, NH₃, glucose, lactate, ²H₂O, acetate, and fatty acids.

i. Protein Precursors

A protein precursor molecule may be any protein precursor molecule known in the art. These precursor molecules may be CO₂, NH₃, glucose, lactate, H₂O, acetate, and fatty acids.

Precursor molecules of proteins may also include one or more amino acids. The precursor may be any amino acid. The precursor molecule may be a singly or multiply deuterated amino acid. For example, the precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted or diluted with non-labeled amino acids. All isotope labeled precursors may be purchased commercially, for example, from Cambridge Isotope Labs (Andover, Mass.).

Protein precursor molecules may also include any precursor for post-translational or pre-translationally modified amino acids. These precursors include but are not limited to precursors of methylation such as glycine, serine or H₂O; precursors of hydroxylation, such as H.sub.2O or O.sub.2; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non-essential amino acid is known—e.g. 4 in alanine, 2 in glycine, etc.

The precursor molecule for proteins may be water. The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—i.e., that the protein is newly synthesized. Analytically, the amino add derivative used must contain all the C—H bonds but must remove all potentially contaminating N—H and O—H bonds.

Hydrogen atoms from body water may be incorporated into free amino acids. ²H or ³H from labeled water can enter into free amino adds in the cell through the reactions of intermediary metabolism, but ²H cannot enter into amino acids that are present in peptide bonds or that are bound to transfer RNA. Free essential amino acids may incorporate a single hydrogen atom from body water into the .alpha.-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutrate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histine, the hydroxyl group in hydroxyproline or hydroxylysine, and others. Other amino adds synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from ¹⁸O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after post-translational modification step (e.g. methylation, hydroxylation, phosphorylation, prenylation, sulfation, carboxylation, acetylation or other known post-translational modifications).

Protein precursors for that are suitable for administration into a subject include, but are not limited to H₂O, CO₂, NH₃ and HCO₃, in addition to the standard amino acids found in proteins.

ii. Organic Metabolite Precursors

Precursors of organic metabolites may be any precursor molecule capable of entering into the organic metabolite pathway. Organic metabolites and organic metabolite precursors include, but are not limited to, H₂O, CO₂, NH₃, HCO₃, amino acids, monosaccharides, carbohydrates, lipids, fatty acids, nucleic acids, glycolytic intermediates, acetic acid, and tricarboxylic acid cycle intermediates. Isotope labeled organic metabolite precursors include, but are not limited to, ²H₂O, ¹⁵NH₃, ¹³CO₂, H¹³CO₃, ²H-labeled amino acids, ¹³C-labeled amino acids, ¹⁵N-labeled amino acids, and ¹⁸O-labeled amino acids.

Organic metabolite precursors may also be administered directly. Mass isotopes that may be useful in mass isotope labeling of organic metabolite precursors include, but are not limited to, ²H, ¹³C, ¹⁴C, ₁₅N, ¹⁷O, and ¹⁸O or other stable isotopes of elements present in organic systems. It is often desirable, in order to avoid metabolic loss of isotope labels, that the isotope-labeled atom(s) be relatively non-labile or at least behave in a predictable manner within the subject. By administering the isotope-labeled precursors to the biosynthetic pool, the isotope-labeled precursors can become directly incorporated into organic metabolites formed in the pool.

iii. Polynucleotide Precursors

Precursors of nucleic acids (i.e., RNA, DNA) are any compounds suitable for incorporation into RNA and/or DNA synthetic pathways. Examples of substrates useful in labeling the deoxyribose ring of DNA include, but are not limited to, [6,6-²H₂] glucose,[U-¹³C₆] glucose and [2-¹³C₁] glycerol (see U.S. Pat. No. 6,461,806). Labeling of the deoxyribose is superior to labeling of the information-carrying nitrogen bases in DNA because it avoids variable dilution sources. The stable isotope labels are readily detectable by mass spectrometric techniques.

In one embodiment, a stable isotope label is used to label the deoxyribose ring of DNA from glucose, precursors of glucose-6-phosphate or precursors of ribose-5-phosphate. In embodiments where glucose is used as the starting material, suitable labels include, but are not limited to, deuterium-labeled glucose such as [6,6-²H₂] glucose, [1-²H₁] glucose, [3-²H₁] glucose, [²H₇] glucose, and the like; ¹³C-1 labeled glucose such as [1-¹³C₁] glucose, [U-¹³C₆] glucose and the like; and ¹⁸O-labeled glucose such as [1-¹⁸O₂] glucose and the like.

In embodiments where a glucose-6-phosphate precursor or a ribose-5-phosphate precursor is desired, a gluconeogenic precursor or a metabolite capable of being converted to glucose-6-phosphate or ribose-5-phosphate can be used. Gluconeogenic precursors include, but are not limited to, ¹³-labeled glycerol such as [2-¹³C₁] glycerol and the like, a ¹³C-labeled amino acid, deuterated water (²H₂O) and ¹³-labeled lactate, alanine, pyruvate, propionate or other non-amino acid precursors for gluconeogenesis. Metabolites which are converted to glucose-6-phosphate or ribose-5-phosphate include, but are not limited to, labeled (²H or ¹³C) hexoses such as [1-²H₁] galactose, [U-¹³C] fructose and the like; labeled (²H or ¹³C) pentoses such as [1-¹³C₁] ribose, [1-²H₁] xylitol and the like, labeled (²H or ¹³C) pentose phosphate pathway metabolites such as [1-²H₁] seduheptalose and the like, and labeled (²H or ¹³C) amino sugars such as [U-¹³C] glucosamine, [1-²H₁] N-acetyl-glucosamine and the like.

The present disclosure also encompasses stable isotope labels which label purine and pyrimidine bases of DNA through the de novo nucleotide synthesis pathway. Various building blocks for endogenous purine synthesis can be used to label purines and they include, but are not limited to, ¹⁵N-labeled amino acids such as [¹⁵N] glycine, [¹⁵N] glutamine, [¹⁵N] aspartate and the like, ¹³C-labeled precursors such as [1-¹³C₁] glycone, [3-¹³C₁] acetate, [¹³C]HCO₃, [¹³C] methionine and the like, and H-labeled precursors such as ²H₂O. Various building blocks for endogenous pyrimidine synthesis can be used to label pyrimidines and they include, but are not limited to, ¹⁵N-labeled amino acids such as [¹⁵N] glutamine and the like, ¹³C-labeled precursors such as [¹³C]HCO₃, [U-¹³C₄] aspartate and the like, and ²H-labeled precursors (²H₂O).

It is understood by those skilled in the art that in addition to the list above, other stable isotope labels which are substrates or precursors for any pathways which result in endogenous labeling of DNA are also encompassed within the scope of the invention. The labels suitable for use in the present invention are generally commercially available or can be synthesized by methods well known in the art.

iv. Water as a Precursor Molecule

In one embodiment, isotope-labeled water may serve as a precursor in the methods described herein. Water is a precursor of proteins, polynucleotides, and many other organic metabolites. As such, labeled water may serve as a precursor in the methods taught herein. H₂O availability is probably never limiting for biosynthetic reactions in a cell (because H₂O represents close to 70% of the content of cells, or >35 Molar concentration), but hydrogen and oxygen atoms from H₂O contribute stochiometrically to many reactions involved in biosynthetic pathways: e.g.: R—CO—CH2—COOH+NADPH+H₂O→R—CH₂CH2COOH (fatty acid synthesis).

As a consequence, isotope labels provided in the form of H- or O-isotope-labeled water are incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabelled water (H₂O) (i.e., by reversal of the same non-enzymatic exchange reactions through which ²H or ³H was incorporated in the first place), however:

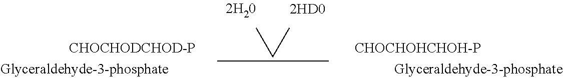

As a consequence, potentially contaminating hydrogen label that does not reflect biosynthetic history, but is incorporated via non-synthetic exchange reactions, can easily be removed in practice by incubation with natural abundance H₂O.

Isotope-labeled water may be readily obtained commercially. “Isotope-labeled water” or “heavy water” includes water labeled with one or more specific heavy isotopes of either hydrogen or oxygen. Specific examples of isotope-labeled water include ²H₂O, ³H₂O, and H₂¹⁸O. For example, ²H₂O may be purchased from Cambridge Isotope Labs (Andover, Mass.), and ³H₂O may be purchased from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 litres water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the present disclosure. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. As long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

v. Carbohydrate Precursors

Compositions comprising carbohydrates may include monosaccharides, polysaccharides, or other compounds attached to monosaccharides or polysaccharides.

Isotope labels may be incorporated into carbohydrates or carbohydrate derivatives. These include monosaccharides (including, but not limited to, glucose and galactose), amino sugars (such as N-Acetyl-Galactosamine), polysaccharides (such as glycogen), glycoproteins (such as sialic acid) glycolipids (such as galactocerebrosides), glycosaminoglycans (such as hyaluronic acid, chondroitin-sulfate, and heparan-sulfate) by biochemical pathways known in the art.

²H-labeled sugars may be administered to an individual as monosaccharides or as polymers comprising monosaccharide residues. Labeled monosaccharides may be readily obtained commercially (e.g., Cambridge Isotopes, Mass.).

Relatively low quantities of compounds comprising ²H-labeled sugars need be administered. Quantities may be on the order of milligrams, 10¹ mg, 10² mg, 10³ mg, 10⁴ mg, 10⁵ mg, or 10⁶ mg. ²H-labeled sugar enrichment may be maintained for weeks or months in humans and in animals without any evidence of toxicity. The lower expense of commercially available labeled monosaccharides, and low quantity that need to be administered, allow maintenance of enrichments at low expense.

In one embodiment, the labeled sugar is glucose. Glucose is metabolized by glycolysis and the citric acid cycle. Glycolysis releases most of the H-atoms from C—H bonds of glucose; oxidation via the citric acid cycle ensures that all H-atoms are released to H₂O. The loss of ²H-label by glucose has been used to assess glycolysis, an intracellular metabolic pathway for glucose. Some investigators have used release of ³H from intravenously administered ³H-glucose into ³H₂O as a measure of glycolysis. Release of ²H-glucose into ²H₂O has not been used previously, because of the expectation that the body water pool is too large relative to ²H administration in labeled glucose to achieve measurable 2H2O levels. In a further variation, the labeled glucose may be [6,6-²H₂]glucose, [1-²H₁] glucose, and [1,2,3,4,5,6-²H₇]glucose.

In another embodiment, labeled sugar comprises fructose or galactose. Fructose enters glycolysis via the fructose 1-phosphate pathway, and secondarily through phosphorylation to fructose 6-phosphate by hexokinase. Galactose enters glycolysis via the galactose to glucose interconversion pathway. Any other sugar is envisioned for use in the present invention. Contemplated monosaccharides include, but are not limited to, trioses, pentoses, hexose, and higher order monosaccharides. Monosaccharides further include, but are not limited to, aldoses and ketoses.

In another embodiment, polymers comprising polysaccharides may be administered. In yet another embodiment, labeled polysaccharides may be administered. In yet another embodiment, labeled sugar monomers may be administered as a component of sucrose (glucose α-(1,2)-fructose), lactose (galactose·β(3-(1,4)-glucose), maltose (glucose α-(1,4)-glucose), starch (glucose polymer), or other polymers.

In one embodiment, the labeled sugar may be administered orally, by gavage, intraperitoneally, intravascularly (e.g., intravenously, intraarterially), subcutaneously, or other bodily routes. In particular, the sugars may be administered to an individual orally, optionally as part of a food or drink.

vi. Lipid Precursors

Measuring the metabolism of compounds comprising ²H-labeled fatty acids are also contemplated by the present invention. Isotope labels from isotope-labeled water may also be incorporated into fatty acids, the glycerol moiety of acyl-glycerols (including but not limited to, triacylglycerides, phospholipids, and cardiolipin), cholesterol and its derivatives (including but not limited to cholesterol-esters, bile acids, steroid hormones) by biochemical pathways known in the art.

Complex lipids, such as glycolipids and cerebrosides, can also be labeled from isotope-labeled water, which is a precursor for the sugar-moiety of cerebrosides (including, but not limited to, N-acetylgalactosamine, N-acetylglucosamine-sulfate, glucuronic acid, and glucuronic acid-sulfate).

²H-labeled fatty acids may be administered to an individual as fats or other compounds containing the labeled fatty acids. ²H-labeled fatty acids may be readily obtained commercially. Relatively low quantities of labeled fatty acids need be administered. Quantities may be on the order of milligrams, 10¹ mg, 10² mg, 10³ mg, 10⁴ mg, 10⁵ mg, or 10⁶ mg. Fatty acid enrichment, particularly with ²H, may be maintained for weeks or months in humans and in animals without any evidence of toxicity. The lower expense of commercially available labeled fatty acids, and low quantity that need to be administered, allow maintenance of enrichments at low expense.

The release of labeled fatty acids, particularly ²H-fatty acid, to labeled water, particularly ²H₂O, accurately reflects fat oxidation. Administration of modest amounts of labeled-fatty acid is sufficient to measure release of labeled hydrogen or oxygen to water. In particular, administration of modest amounts of ²H-fatty acid is sufficient to measure release of ²H to deuterated water.

In another variation, the labeled fatty acids may be administered orally, by gavage, intraperitoneally, intravascularly (e.g., intravenously, intraarterially), subcutaneously, or other bodily routes. In particular, the labeled fatty acids may be administered to an individual orally, optionally as part of a food or drink.

ii. Modes of Administering Precursors

Modes of administering the one or more isotope-labeled precursor molecules may vary, depending upon the absorptive properties of the isotope-labeled precursor and the specific biosynthetic pool into which each compound is targeted. Precursors may be administered to organisms, plants and animals including humans directly for in vivo analysis. In addition, precursors may be administered in vitro to living cells. Specific types of living cells include hepatocytes, adipocytes, myocytes, fibroblasts, neurons, pancreatic β-cells, intestinal epithelial cells, leukocytes, lymphocytes, erythrocytes, microbial cells and any other cell-type that can be maintained alive and functional in vitro.

Generally, an appropriate mode of administration is one that produces a steady state level of precursor within the biosynthetic pool and/or in a reservoir supplying such a pool for at least a transient period of time. Intravenous or oral routes of administration are commonly used to administer such precursors to organisms, including humans. Other routes of administration, such as subcutaneous or intra-muscular administration, optionally when used in conjunction with slow release precursor compositions, are also appropriate. Compositions for injection are generally prepared in sterile pharmaceutical excipients. Modes of administration may comprise continuous administration or discontinuous administration (e.g., a pulse chase).

In some embodiments, the one or more isotope-labeled precursor molecules may be administered to a human. In other embodiments, the one or more isotope-labeled precursor molecules may be administered to a veterinary or research subject, including without limitation mammals such as rodents, primates, hamsters, guinea pigs, horses, dogs, or pigs.

B. Obtaining a Biological Sample

A plurality of molecules of interest may be acquired from the cell, tissue, or organism. The one or more biological samples may be obtained, for example, by blood draw, urine collection, biopsy, or other methods known in the art. The one or more biological sample may be one or more biological fluids. The molecule of interest may also be obtained from specific organs or tissues, such as muscle, liver, adrenal tissue, prostate tissue, endometrial tissue, blood, skin, and breast tissue. Molecules of interest may be obtained from a specific group of cells, such as tumor cells or fibroblast cells. Molecules of interest also may be obtained, and optionally partially purified or isolated, from the biological sample using standard biochemical methods known in the art.

A sample may include a tissue histology specimen from tissues such as, for example, the gut, skin, organs, breast, prostate, brain, bone, muscle, liver, and gut. The sample may also be obtained from bodily fluids including, for example, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, and intestinal secretions. The sample may further include biofilms, microbiomes and other microbial organisms. The sample may be a clinical sample, upon which a clinical decision, diagnosis or prognosis can be made using the output generated according to the methods described herein.

The sample may be obtained, for example, by blood draw, urine collection, biopsy, or other methods known in the art. In some embodiments, the sample is obtained by taking a surgical biopsy; surgical removal of a tissue or portion of a tissue; performing a percutaneous, endoscopic, transvascular, radiographic-guided or other non-surgical biopsy; euthanizing an experimental animal and removing tissue; collecting ex vivo experimental preparations; removing tissue at post-mortem examination; or other methods known in the art for collecting tissue samples. The methods of obtaining a sample may also vary and be specific to the molecules of interest.

Standard techniques for preparing a sample for mass spectrometry include, for example, freezing and slicing, lyophilization, cryopreservation, ethanol dehydration, OCL preservation, and other suitable methods known in the art. In some embodiments, the samples are prepared on a slide with a coated surface that permits or increases energy-dependent volatilization of molecules from the surface of the slide.

The frequency of biological sampling can vary depending on different factors. Such factors include, but are not limited to, the nature of the molecules of interest, ease and safety of sampling, synthesis and breakdown/removal rates of the molecules of interest, and the half-life of a compound (chemical entity, biological factor, already-approved drug, drug candidate, drug lead, etc.).

C. Enriching or Isolating a Stable Isotope-Labeled Molecule of Interest

In some embodiments, a stable isotope-labeled target molecule of interest is enriched or isolated from a biological sample. Polynucleotides, polypeptides, or other organic metabolites may be partially purified, enriched, or isolated, from a biological sample using standard biochemical methods known in the art. For example, suitable methods of enriching or isolating a protein may include, but are not limited to, immunoprecipitation, chromatography (e.g., by size exclusion, hydrophobic interaction, affinity, metal binding, immunoaffinity, or HPLC), centrifugation through a density gradient, etc. Suitable methods for enrichment and isolation may depend upon, for example, abundance of the molecule of interest, biochemical properties of the molecule of interest, the type of sample, and the relative degree of enrichment or purity required.

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the molecules of interest. The molecules of interest also may be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

In some embodiments, the one or more target molecules of interest may be peptides or amino acids isolated or derived from one or more polypeptides. In other embodiments, the one or more target molecules of interest may be deoxyribose molecules isolated or derived from DNA. In certain embodiments, the isotopologues (e.g., a first and a second isotopologue of the present disclosure) on which the high resolution mass spectrometric measurement is performed are derived from a fragment ion, the fragment ion being derived from the one or more stable isotope-labeled target molecules of interest. In some embodiments, the one or more target molecules of interest are isolated from a cell, e.g., using immunoprecipitation, chromatography (e.g., by size exclusion, hydrophobic interaction, affinity, metal binding, immunoaffinity, or HPLC), centrifugation through a density gradient, and so forth.

D. Performing a High Resolution Mass Spectrometric Measurement

Isotopic enrichment in proteins and organic metabolites can be determined by various mass spectrometric methods. Mass spectrometers convert molecules such as polynucleotides, polypeptides, or other organic metabolites into rapidly moving gaseous ions and separate them on the basis of their mass-to-charge ratios. The distributions of isotopes or isotopologues of ions, or ion fragments, may thus be used to measure the isotopic enrichment in a plurality of polynucleotides, polypeptides, or other organic metabolites. Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers. Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization. Different ionization methods are also known in the art. One key advance has been the development of techniques for ionization of large, non-volatile macromolecules including proteins and polynucleotides. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, two or more mass analyzers may be coupled (MS/MS) first to separate precursor ions, then to separate and measure gas phase fragment ions. These instruments generate an initial series of ionic fragments of a protein, and then generate secondary fragments of the initial ions. The resulting overlapping sequences allows complete sequencing of the protein, by piecing together overlaying “pieces of the puzzle” based on a single mass spectrometric analysis within a few minutes (plus computer analysis time).

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS (or other mass spectrometric modalities that analyze ions of proteins and organic metabolites, rather than small inorganic gases) is used to measure mass isotopomer abundances of organic molecules, hydrogen-labeled isotope incorporation from isotope-labeled water is amplified 3 to 7-fold, depending on the number of hydrogen atoms incorporated into the organic molecule from isotope-labeled water in vivo.

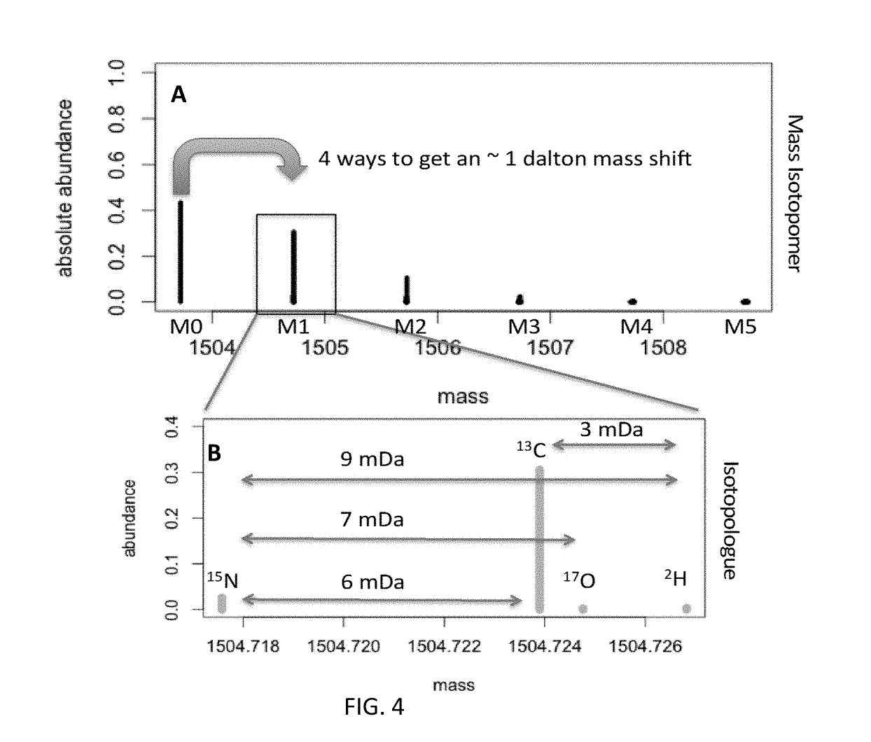

Certain aspects of the present disclosure relate to high resolution mass spectrometric measurements. As described herein, high resolution mass spectrometric measurements allow the resolution of one or more isotopologues within the same mass isotopomer, such as ¹⁵N, ¹³C, ¹⁷O, ¹⁸O, and/or ²H-labeled isotopologues in the same mass isotopomer (e.g., an M1 mass isotopomer). In some embodiments, a high resolution mass spectrometric measurement may refer to a measurement performed by a high resolution mass spectrometer capable of quantifying isotopologues that differ in mass by about 9 or fewer mDa, about 8 or fewer mDa, about 7 or fewer mDa, about 6 or fewer mDa, about 5 or fewer mDa, about 4 or fewer mDa, about 3 or fewer mDa, about 2 or fewer mDa, or about 1 or fewer mDa. In some embodiments, a high resolution mass spectrometric measurement may refer to a measurement performed by a high resolution mass spectrometer capable of quantifying isotopologues that differ in exact mass but share the same nominal mass. In some embodiments, a high resolution mass spectrometric measurement may refer to a measurement performed by a high resolution mass spectrometer capable of quantifying and resolving one or more isotopologues depicted in FIGS. 4A and 4B.

In some embodiments, the high resolution mass spectrometer is a Fourier transform-based mass spectrometer, including without limitation a Fourier transform ion cyclotron resonance (FT-ICR) mass spectrometer such as an Orbitrap mass spectrometer. In FT-ICR, as is known in the art, ions are trapped in a Penning trap and excited at resonant cyclotron frequencies through circular orbits dictated by the Lorentz forces generated between a magnetic field and an orthogonal electric field. In Orbitrap, a barrel-like outer electrode at ground potential and a spindle-like central electrode are used to trap ions in trajectories rotating elliptically around the central electrode with oscillations along the central axis, confined by the balance of centrifugal and electrostatic forces. The use of such instruments employs a Fourier transform operation to convert a time domain signal (e.g., frequency) from detection of image current into a high resolution mass measurement. Further descriptions and details may be found, e.g., in Scheltema, R. A. et al. (2014) Mol. Cell Proteomics 13:3698-3708; Perry, R. H. et al. (2008) Mass. Spectrom. Rev. 27:661-699; and Scigelova, M. et al. (2011) Mol. Cell Proteomics 10:M111.009431.

In general, in order to determine a control isotopologue frequency distribution for a molecule of interest (e.g., a control relative abundance), a sample is taken before infusion of an isotopically labeled precursor. Such a measurement is one means of establishing in the cell, tissue or organism, the naturally occurring frequency of isotopologues of the molecule. When a cell, tissue or organism is part of a population of subjects having similar environmental histories, a population isotopologue frequency distribution may be used for such a background measurement. Additionally, such a control isotopologue frequency distribution may be estimated, using known average natural abundances of isotopes. For example, in nature, the natural abundance of ¹³C present in organic carbon in 1.11%. Methods of determining such isotopologue frequency distributions are discussed below. Typically, samples of the molecule of interest are taken prior to and following administration of an stable isotopically labeled precursor molecule to the subject and analyzed for isotopologue frequency as described below.

E. Isotopologue Abundances

The high resolution mass spectrometric measurement techniques described above are used to measure isotopologue abundances, e.g., within the same mass isototopomer, or from different mass isotopomers. In some embodiments, abundance may refer to peak height on a mass spectrum. In some embodiments, abundance may refer to area under the peak. In some embodiments, a relative abundance may be expressed as a ratio toward the parent (e.g., all ¹²C) mass isotopomer, as described below. It is appreciated that any calculation means which provides values for the abundances of isotopologues in a sample may be used in describing such data, for the purposes of the present disclosure.

In some embodiments, abundance of two or more isotopologues may be measured. Any pair of isotopologues, including ²H-, ¹³C-, ¹⁵N-, and ¹⁷O-isotopologues may be measured and used in calculations described herein. In certain embodiments, the abundances of a ²H-isotopologue and a ¹³C-isotopologue may be measured. In certain embodiments, the abundances of a ²H-isotopologue and a ¹³C-isotopologue in the same mass isotopomer may be measured.

In some embodiments, the two or more isotopologues represent two distinct exact masses in the same mass isotopomer, e.g., having different exact masses of a mass difference resolvable by high resolution mass spectrometry. In some embodiments, the two or more isotopologues represent two distinct exact masses in an M1 mass isotopomer. In certain embodiments, the relative abundances of a ²H-isotopologue and a ¹³C-isotopologue in an M1 mass isotopomer may be measured.

A relative abundance of an isotopologue may then be calculated, based at least in part on the abundances of two or more isotopologues. A variety of methods useful for calculating a relative abundance of an isoptologue would be apparent to one of skill in the art. In some embodiments, a relative abundance of an isotopologue of interest may be expressed as a ratio of the abundance of an isotopologue of interest (e.g., a ²H-labeled isotopologue from the M1 mass isotopomer) to the sum of the abundance of the isotopologue of interest and the abundance of a second isotopologue having a different exact mass in the same mass isotopomer (e.g., a ¹³C-labeled isotopologue from the M1 mass isotopomer). Without wishing to be bound to theory, it is thought that the large increase in signal intensity of, e.g., a ²H-labeled isotopologue over its naturally occurring form, coincident with the very small mass difference between the ¹³C-isotopologue and the ²H-isotopologue in the M1-mass isotopomer (2.9 millidaltons) after isotope labeling, favors the use of high resolution mass spectrometry, in which ions with highly similar masses are subject to fewer potential biases (e.g., different behavior of ions in the trap as a function of their mass) than those with more dissimilar masses.

In other embodiments, high resolution mass spectrometric measurement may be used to measure abundances of isotopologues in different mass isotopomers. Without wishing to be bound to theory, it is thought that such methods may yield less background signal, e.g., with a deuterium or ¹⁵N label, than in a mass isotopomer (which includes naturally abundant ¹³C). For example, a leucine with 3 deuterium labels (d3-leucine) may be administered, and an abundance of a 3×²H-isotopologue of the M3 mass isotopomer may be measured and compared relative to a ¹³C isotopologue in the M0 isotopomer, instead of measuring the M3 and M0 mass isotopomers by traditional lower resolution mass spectrometric measurements. In other applications, however, particularly with administration of heavy water, it is thought that comparing isotopologues in the M2 or M3 mass isotopomers may be more complicated than staying within the M1 mass isotopomer—the higher mass isotopomers contain combinations of ²H, ¹³C, and other isotopologues, which smears out their capacity to be separated.

In some embodiments, a relative abundance of an isotopologue of interest may be expressed as a ratio of the abundance of an isotopologue of interest (e.g., a ²H-labeled isotopologue from the M1 mass isotopomer) to the abundance of a second isotopologue having a different exact mass in the same mass isotopomer (e.g., a ¹³C-labeled isotopologue from the M1 mass isotopomer). In other embodiments, a relative abundance of an isotopologue of interest may be expressed as a ratio of the abundance of an isotopologue of interest (e.g., a 3×²H-isotopologue of the M3 mass isotopomer) to the abundance of a second isotopologue from a different mass isotopomer (e.g., a ¹³C isotopologue in the M0 isotopomer).

In some embodiments, a relative abundance of an isotopologue of interest may be expressed as a ratio of the abundance of an isotopologue of interest (e.g., a ²H-labeled isotopologue from the M1 mass isotopomer) to the sum of the abundance of the isotopologue of interest and the abundances of a multiple other isotopologues having different exact masses in the same mass isotopomer (e.g., a ¹³C-labeled isotopologue and a ¹⁵N-labeled isotopologue from the M1 mass isotopomer). In other embodiments, a relative abundance of an isotopologue of interest may be expressed as a ratio of the abundance of an isotopologue of interest (e.g., a 3×²H-isotopologue of the M3 mass isotopomer) to the sum of the abundance of the isotopologue of interest and the abundances of a multiple other isotopologues from different mass isotopomers (e.g., a ¹³C isotopologue in the M0 isotopomer and a ¹³C-labeled isotopologue in the M1 mass isotopomer).

In some embodiments, the abundances of a first isotopologue and a second isotopologue are of comparable peak heights or signal intensities. Without wishing to be bound to theory, it is thought that this may reduce the potential analytic impact of non-linearities of detector quantitation or differences in ion counting statistics when quantifying isotopologue abundances. In some embodiments, “comparable” peak heights or signal intensities may refer to peak heights or signal intensities where the total abundance values are no more than about 50%, no more than about 40%, no more than about 30%, no more than about 25%, no more than about 20%, or no more than about 10% different from each other.

In some embodiments, the methods described herein allow the high resolution mass spectrometric measurement of a change in the abundances of two or more isotopologues from the same mass isotopomer even if the abundance of the mass isotopomer as a whole changes a slight amount or not at all. This may be the case when distinct isotopologues are entering and leaving a mass isotopomer simultaneously, which may occur with significant molecular flux rates even if the overall mass isotopomer abundance does not change. For example, ²H-isotopologues may enter an M1 mass isotopomer while ¹³C-isotopologues are leaving, resulting in significant molecular flux rates with only a slight change, or no change, in the relative abundance of the M1 mass isotopomer itself. Advantageously, the methods of the present disclosure allow for ways to measure these molecular flux rates, which would be invisible to techniques that only measure mass isotopomer abundance. In some embodiments, a small or no change in relative abundance of a mass isotopomer may refer to a change in theoretical maximal relative abundance that is less than about 5% of the total mass isotopomer envelope measured or to a change in measurable or observable relative abundance that is less than about 1% of the total mass isotopomer envelope measured.

F. Calculations

In some embodiments, based on measurements of isotopologue abundances as described above, measured excess molar ratios may be calculated for isolated isotopologue species of a molecule. The practitioner then compares measured internal pattern of excess ratios to the theoretical patterns. Such theoretical patterns can be calculated using the binomial or multinomial distribution relationships as described in U.S. Pat. Nos. 5,338,686; 5,910,403; and 6,010,846. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. Pat. No. 7,001,587. In addition to the above-cited references, calculation software implementing the method is publicly available from Professor Marc Hellerstein, University of California, Berkeley.

Relative abundance is then calculated. For example and without limitation, a relative abundance of a ²H-isotopologue may be expressed as a comparison with the abundance of a ¹³C isotopologue in the M1-mass isotopomer of a molecule, as described above. Relative abundance of the ²H-isotopologue may be calculated from measured peak heights by the equation:

$$Relative abundance {}^{2} HM 1 = \frac{Abundance {}^{2} HM 1}{( Abundance {}^{2} HM 1 + Abundance {}^{13} CM 1 )}$$

where Abundance ²HM1 refers to the measured peak height of the ²H isotopologue in the M1-mass isotopomer, and Abundance ¹³ CM1 refers to the measured peak height of the ¹³C isotopologue in the M1-mass isotopomer. A further example of calculating relative abundances from peak heights is provided in Table 2 below.

Relative abundance of an isotopologue of interest may then be compared to a control relative abundance. In some embodiments, a control relative abundance may refer to a relative abundance (e.g., any expression of relative abundance described herein) of an isotopologue as it naturally occurs, e.g., without administration of an exogenous isotope label. In some embodiments, a control relative abundance may refer to a relative abundance (e.g., any expression of relative abundance described herein) of the same type of target molecule of interest from which the experimental isotopologue was derived but taken from a sample collected prior to administration of the stable isotope-labeled precursor molecule. In some embodiments, a control relative abundance may refer to a relative abundance (e.g., any expression of relative abundance described herein) of the same type of target molecule of interest from which the experimental isotopologue was derived but taken from a sample collected without administration of the stable isotope-labeled precursor molecule. In some embodiments, a control relative abundance may refer to a known, calculated, or expected relative abundance (e.g., any expression of relative abundance described herein) based on the natural abundance of an isotope or isotopes within a particular isotopologue.

In some embodiments, comparing the relative abundance of an isotopologue to a corresponding control relative abundance may refer to a determination of enrichment or depletion (excess relative abundance, which may be a positive or negative number, respectively) during or after administration of the stable isotope-labeled precursor. e.g.,

$${( Relative {abundance}_{x} )}_{e} - {( Relative {abundance}_{x} )}_{b} = {( \frac{Abundance {}^{2} HM 1}{( Abundance {}^{2} HM 1 + Abundance {}^{13} CM 1 )} )}_{e} - {( \frac{Abundance {}^{2} HM 1}{( Abundance {}^{2} HM 1 + Abundance {}^{13} CM 1 )} )}_{b} ,$$

where subscript e refers to enriched and b refers to baseline or natural abundance.

In order to determine the fraction of stable isotope-labeled molecules of interest that were actually newly synthesized during a period of precursor administration, the measured excess molar ratio (EM_{X}) is compared to the calculated theoretical maximum enrichment value, A_{X}*, which describes the enrichment in 100% newly synthesized stable-isotope labeled molecules of interest for an isotopologue, to reveal the isotopologue excess ratio which would be expected to be present if all isotopologues were from newly synthesized molecules assembled during the period of exposure to label (maximal Δ relative abundance). In some embodiments, a method of determining rate of synthesis includes calculating the proportion of mass isotopically labeled subunits present in the precursor pool, and using this proportion to calculate an expected frequency of molecules containing different numbers of mass isotopically labeled subunits. These expected frequencies (maximal Δ relative abundances) are then compared to the actual, experimentally determined isotopologue frequencies (observed Δ relative abundances). From these values, the proportion or fraction of the molecules of interest which were synthesized during a selected label incorporation period can be determined. Thus, the rate of synthesis, calculated as fraction or absolute mass of new molecules synthesized per unit of time, during such a time period is also determined. In another embodiment, a fully replaced (100% labeled) reference molecule is isolated from a subject and used as an internal standard to calculate the fractional synthesis of a molecule of interest over a time period. The excess relative abundance of an isotopologue in the fully replaced reference molecule (maximal Δ relative abundance) is used as the denominator and the excess relative abundance of an isotopologue in the molecule (observed Δ relative abundance) of interest is used as the numerator in the equation to calculate the proportion or fraction of the molecules of interest which were synthesized during a selected label incorporation period.

In these embodiments, the fraction of newly synthesized target molecules of interest may be calculated based on equations known in the art that involve a comparison of the relative abundance of an isotopologue and a corresponding control relative abundance of the isotopologue in a molecule of interest to a maximal relative abundance of the isotopologue in a fully replaced reference molecule, e.g., as described above. Equations for determining a fraction of newly synthesized target molecules of interest during or after a period of stable isotope label administration are well known in the art and include without limitation:

$${( Fraction newly synthesized )}_{i} = \frac{{( observed Δ Relative {abundance}_{x} )}_{i}}{{( maximal Δ Relative {abundance}_{x} )}_{i}}$$

where the maximal ΔRelative abundance for isotopologue x is calculated based on the labeling conditions present in a subject or sample during or after administration of a stable isotope-labeled precursor molecule for a period of time i.

In some embodiments, this calculation is derived from the measured or inferred isotopic abundance of the stable isotope-labeled precursor molecule from which the target molecules of interest were synthesized in the subject at the site of biosynthesis of the target molecules of interest, e.g., the calculated theoretical maximum enrichment value, A_{X}*, which describes the enrichment in 100% newly synthesized stable-isotope labeled molecules of interest for an isotopologue, using equations known in the art (see, e.g., Hellerstein, M. K. and Neese, R. A. (1999) Am. J. Physiol. 276:E1146-1170; and Busch, R. et al. (2007) Nat. Protoc. 2:3045-3057).

In some embodiments, precursor-product relationship may then be applied. For the continuous labeling method, the isotopic enrichment is compared to asymptotic (i.e., maximal possible) enrichment and kinetic parameters (e.g., synthesis or replacement rates) are calculated from precursor-product equations. A fractional synthesis rate (kₛ) may be determined by applying the continuous labeling, precursor-product formula:

kₛ=[−In(1−f)]/t

where f=fraction newly synthesized=product enrichment/asymptotic precursor/enrichment (e.g., as described above); t=time of label administration of contacting in the system studied; and the units of replacement rate are fraction per unit of time.

In some embodiments, more than one biological sample may be obtained at different points following administration of a stable isotope-labeled precursor. In this way, a fraction of newly synthesized target molecules of interest may be calculated for the period between administration of the stable isotope-labeled precursor and obtaining the first sample, between administration of the stable isotope-labeled precursor and obtaining the second sample, and/or between obtaining the first sample and obtaining the second sample. In some embodiments, such calculations may be used to calculate observed fractional synthesis from the change in relative abundance of an isotopologue by comparison to the theoretical maximum change in relative abundance of an isotopologue at a particular percentage or level of enrichment.

In some embodiments, a rate of breakdown or degradation of the target molecules of interest may be calculated based on a comparison of the relative abundance of an isotopologue and the control relative abundance of the isotopologue, e.g., as described above. Equations for determining a rate of breakdown or degradation of the target molecules of interest during or after a period of stable isotope label administration are well known in the art. For example, in a discontinuous labeling method, the rate of decline in isotope enrichment is calculated and the kinetic parameters of the molecules of interest are calculated from exponential decay equations.

Breakdown rate constants (k_{d}) may be calculated based on an exponential or other kinetic decay curve:

k_{d}=[−In f]/t

Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

In some embodiments, one or more of the calculations and/or methods described herein may be used as a diagnostic test. Without wishing to be bound to theory, it is thought that applying the methods described herein may improve the utility of any diagnostic test using a non-radioactive, stable isotopic tracer, and these methods may be particularly advantageous for application in heavy water (²H₂O) labeling protocols, e.g., when changes in mass isotopomer abundances are relatively modest (such as for low dose or brief duration ²H₂O labeling protocols). For example, ²H₂O labeling may be used to measure mass isotopomer abundances (MIAs) and mass isotopomer distributions (MIDs) in the kinetic analysis of lipids (Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286:E577-588), intermediary metabolites (Neese, R. A. et al. (1995) J. Biol. Chem. 270:14452-14466; Hellerstein, M. K. et al. (1997) Am. J. Physiol. 272:E163-172; Hellerstein, M. K. et al. (1997) J. Clin. Invest. 100:1305-1309; and Louie, K. B. et al. (2013) Sci. Rep. 3:1656), proteins (Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814) and cells (Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057). Exemplary molecules of interest and pathological states for which the methods described herein may find use in diagnostic applications include without limitation those described in U.S. Pat. No. 8,005,623.

In some embodiments, a diagnostic test may be used in the diagnosis, management, or treatment selection of a human patient. In other embodiments, a diagnostic test may be used in the diagnosis, management, or treatment selection of a veterinary patient or research subject, including without limitation mammals such as rodents, primates, hamsters, guinea pigs, horses, dogs, or pigs. For example, in some embodiments, the molecular flux rates in one or more metabolic pathways of interest may contribute to the prognosis, survival, morbidity, mortality, stage, therapeutic response, symptomology, disability or other clinical factor of the disease of interest. In one embodiment, the molecular flux rates in the one or more metabolic pathways being measured may be relevant to an underlying molecular pathogenesis, or causation of, one or more diseases. In another embodiment, the molecular flux rates in one or more metabolic pathways of interest may contribute to the initiation, progression, severity, pathology, aggressiveness, grade, activity, disability, mortality, morbidity, disease sub-classification or other underlying pathogenic or pathologic feature of the disease of interest. In some embodiments, one can quantitate the molecular flux rates of one or more molecules of interest within one or more targeted metabolic pathways and use the information as a biomarker of medical diagnosis, prognosis, or therapeutic efficacy of drug or combination drug treatment.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Rationale

As discussed supra, the inability of the current generation of mass spectrometers, such as quadrupole, ion trap, and time-of-flight (ToF) instruments, has helped prevent broad or routine application of mass spectrometric kinetic analysis in clinical medicine and drug development. These instruments have not provided sufficient analytic precision and accuracy for mass isotopomer abundances (MIAs) and mass isotopomer distributions (MIDs) to allow routine application of molecular kinetic methods in clinical medicine or in drug development settings.

As an illustrative example of this problem, a quadrupole GC/MS can routinely achieve reproducibility for MIA quantitation of ±˜0.1-0.2% (±0.001-0.002 fractional abundance) for each mass isotopomer in an isotope envelope of a typical small molecular ion (e.g., m/z<800). (±0.2-0.5%). A different type of instrument, for example a Q-ToF, can achieve analytic accuracy and reproducibility of a similar magnitude, e.g., ˜0.2-0.3% reproducibility and accuracy of 0.2-0.5% (Wolfe, R. R. and Chinkes, D. L. (2004) Isotope Tracers in Metabolic Research: Principles and Practice of Kinetic Analysis, 2^{nd} ed. Wiley; Hellerstein, M. K. and Neese, R. A. (1992) Am. J. Physiol. 263:E988-1001; Hellerstein, M. K. and Neese, R. A. (1999) Am. J. Physiol. 276:E1146-1170; Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814).

This analytic performance translates into the following labeling protocol requirements in human subjects, using heavy water (²H₂O) labeling. A convenient simulation with a fairly typical ca.1.0% enrichment of ²H₂O in body water in humans is shown in Table 1, with the analyzed molecule being 600 daltons and containing 6 potential deuterium labeling sites (k refers to the turnover rate constant).

TABLE 1
Label Incorporation Kinetics in Relation to Analytic Detection
Limits (for a molecule of ~600 daltons with 6 potential labeling
sites at body water enrichment of 1.0%).

| | % Labeled EM1 | % Labeled EM1 | % Labeled EM1 |
|---|---|---|---|
| k (%/day) | (day 1) | (day 5) | (day 10) |
| 1 | 0.03 | 0.15 | 0.30 |
| 2 | 0.06 | 0.30 | 0.60 |
| 5 | 0.15 | 0.75 | 1.50 |

As is apparent from Table 1, detection of sufficient label incorporation into a molecule of mass ˜600 Da with turnover rate between 1%-5%/day (i.e., half-life of 2 weeks to ˜2 months) requires at least several days of label exposure at 1% body water enrichment to achieve label incorporation in the targeted molecule that is adequate in terms of signal to noise ratio for reliable quantitation—i.e. enrichment values that are several times the limit of accurate detection of 0.2-0.3% EM1 and therefore at least 0.50-1.0%. Analytic improvements that allowed the use of a clinical labeling protocol that is substantially less burdensome and less expensive (e.g., a protocol that involves giving a few oral doses of heavy water, ²H₂O, over 1-2 days) would represent a major practical advance for applying ²H₂O labeling methods in medical diagnostics and drug development.

The most powerful class of commercial mass spectrometers in terms of mass resolution, mass measurement accuracy, sensitivity, and analytical breadth and depth for detection and identification of molecules are the FT-ICR instruments, such as the Orbitrap (Scheltema, R. A. et al. (2014) Mol. Cell Proteomics 13:3698-3708). It would therefore be ideal for general kinetic applications if this detection power could be combined with a high degree of accuracy and reproducibility for MIA and MID measurements. Unfortunately, measurement of MIAs and MIDs with FT-ICR has consistently been found to be even less accurate and reproducible than is routinely achieved by much less powerful instruments, such as ToF or quadrupole instruments (Erve, J. C. et al. (2009) J. Am. Soc. Mass Spectrom. 20:2058-2069; and Mathur, R. and O'Connor, P. B. (2009) Rapid Commun. Mass. Spectrom. 23:523-529).

Although the full physical explanations behind this are not understood, there are a number of possible reasons that have been proposed for such poor quantitative performance for MIAs and MIDs by FT-ICR instruments. One potential reason is the well understood, mass-dependent differential decay rates of transients for ions within the trap (Erve, J. C. et al. (2009) J. Am. Soc. Mass Spectrom. 20:2058-2069; and Mathur, R. and O'Connor, P. B. (2009) Rapid Commun. Mass. Spectrom. 23:523-529). This phenomenon relates to different behavior of ions in the trap that is a function of their mass, as well as on their intensity or abundance. Differential decay rates of ions result in biases in the proportion of ions that remain in the trap and are detected. The result of these biases is the inaccurate measurement of true isotope ratios for the ions that entered the trap. It has been observed that the greater the differences in mass or in abundance, the greater the potential biasing effect on measured relative abundances (Perry, R. H. et al. (2008) Mass. Spectrom. Rev. 27:661-699; and Scheltema, R. A. et al. (2014) Mol. Cell Proteomics 13:3698-3708).

Another potential explanation for poor quantitative performance for MIAs and MIDs by mass spectrometers in general is the contamination of mass isotopomers in ions putatively representing an identified target molecule by other ions derived from the biologic matrix in which the target molecular was measured. Random or systematic background “multiplexing” contaminants are in principle especially problematic for molecules that themselves are of low abundance in a biologic matrix, which is often the case and is indeed an advantage of sensitive mass spectrometers such as FT-ICR instruments. In addition, an explanation for the insufficient analytic sensitivity of mass spectrometers in general, including FT-ICR instruments, for quantitation of very small changes in MIAs and MIDs after low doses of isotopic precursors such as ²H₂O relates to the issue of relative change in analytic signal. For mass isotopomers, the natural fractional abundance of the M0, M1, M2 etc. mass isotopomers is typically a much larger number than the perturbation in mass isotopomer fractional abundance after introduction of small amounts of ²H after metabolic labeling with a low dose of ²H₂O. The signal:noise characteristics of such measurements is not optimal for reproducible measurements.

These limitations have prevented the broad application of these exquisitely sensitive and powerful instruments for development of kinetic biomarkers in clinical medicine and biomedical research.

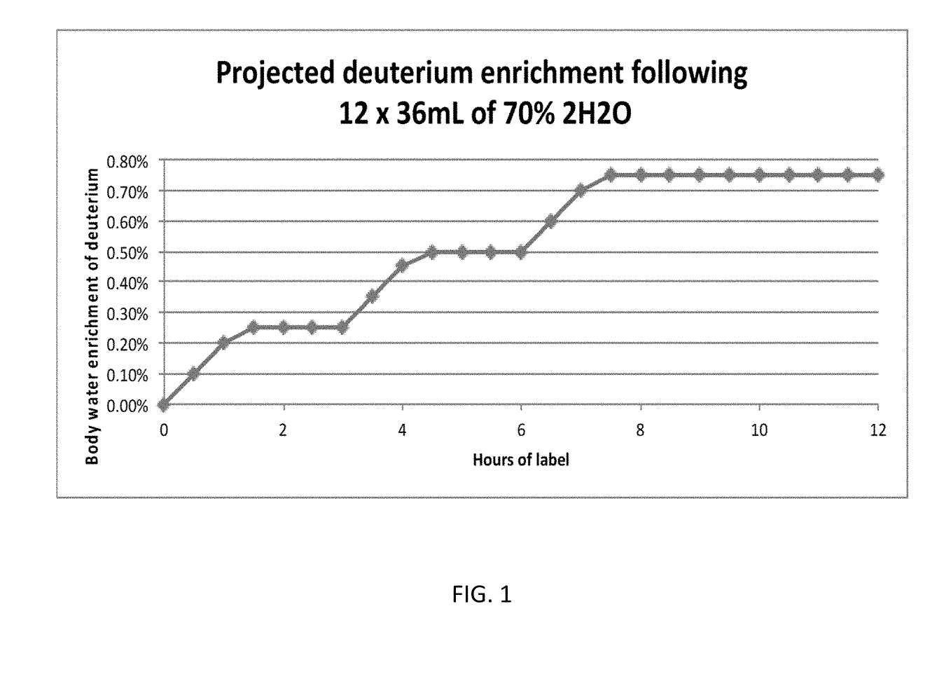

An example of the practical clinical consequences of the current limitations on analytic reproducibility and a potential solution is illustrated in FIGS. 1-3. These figures exemplify a standard low-dose ²H₂O-labeling protocol in which a healthy volunteer takes 4 drinks (50 ml @ 70% ²H₂O) of ²H₂O a day for 3 days. As shown in FIG. 1, this protocol results in a projected deuterium body water enrichment that plateaus at approximately 75% (p), with an average or effective p of 0.5%.

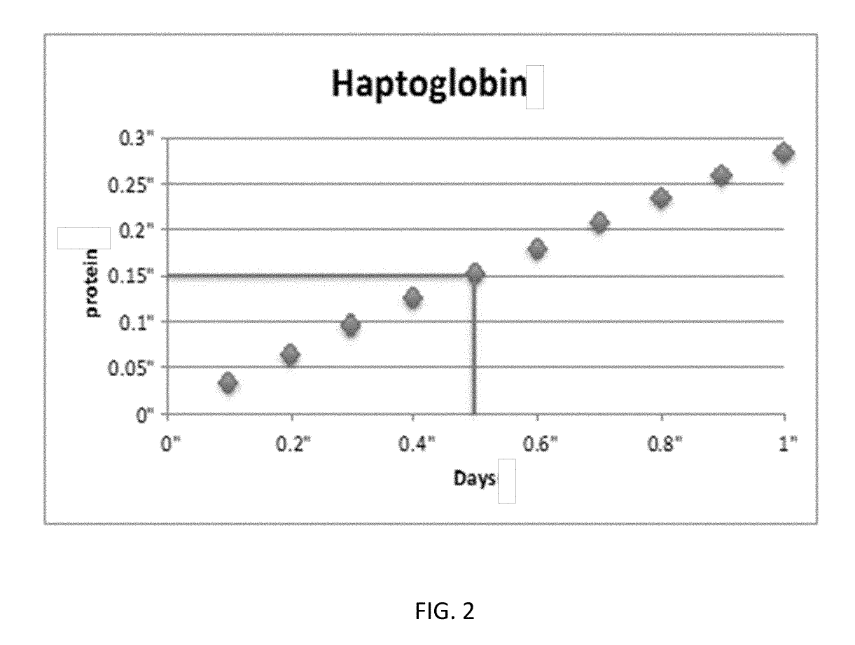

An exemplary protein, plasma haptoglobin, is known to have a fractional replacement rate of approximately 33% per day. FIG. 2 shows that, following one half day of water labeling with the protocol shown in FIG. 1, ˜15% of the molecules present are newly synthesized. Haptoglobin peptides should therefore have a maximal abundance in the most sensitive mass isotopomer EM0 (EM0*) of 0.055 at a p of 0.75%, and the expected change in EM0 is 15% newly synthesized ×0.055 EM0*=0.00825, or 0.825%. The analytic uncertainty in EM0 on a Time of Flight mass spectrometer is ˜+/−0.50% EM0, so that the uncertainty in measured EM0 is +1-60% of the biologic value present (0.50%/0.825%), resulting in a far too large uncertainty in calculated fractional synthesis measurements to be useful in medical practice.

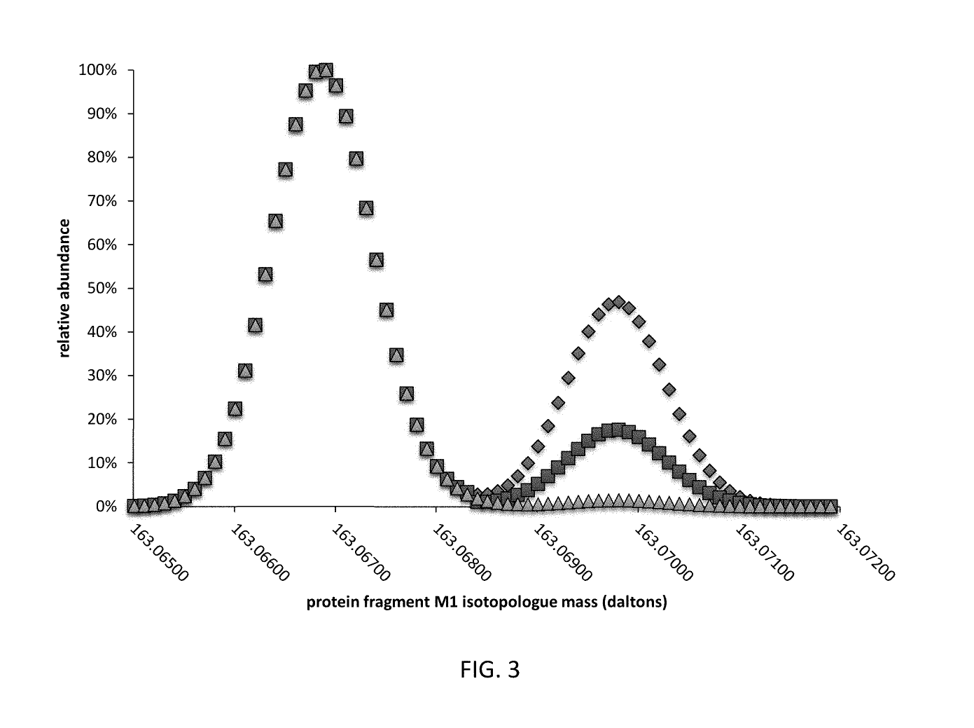

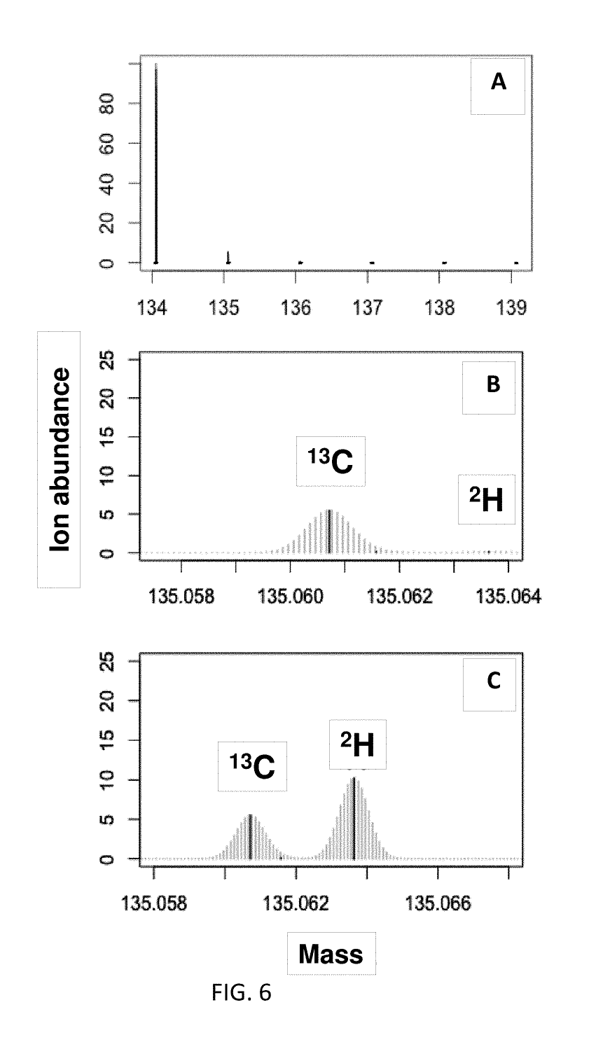

In contrast, as shown in FIG. 3, for 15% newly synthesized haptoglobin molecules, a serine-derived fragment ion from a sample with a p of 0.75% exhibits a ²H% M1-isotopologue abundance of ˜20% relative to the ¹³C M1-isotopologue (see, e.g., FIG. 4B) with essentially no background abundance prior to labeling (see, e.g., FIG. 6C). In principle, the measurement of isotopologues in the M1-mass isotopomer thereby substantially improves analytic sensitivity and reproducibility, compared to measurement of mass isotopomer abundances.

Building on these concepts, an ideal solution to the problem of measuring molecular flux rates in humans and pre-clinical models would have the following features:
1) Take advantage of the high mass resolution, mass measurement accuracy and sensitivity of FT-ICR instruments;
2) Have better accuracy and precision than current FT-ICR measurements (and other current mass spectrometric measurements) of MIA and MID measurements that are based on mass isotopomer abundances, by reducing the impact of differences in decay rates of transients due to different mass-to-charge ratios and different intensities of ions;
3) Be less susceptible to interferences from contaminating ions in complex analytic matrices;
4) Have better accuracy and precision than current FT-ICR measurements (and other current mass spectrometric measurements) of MIA and MID measurements, e.g.,
   (i) by increasing the relative change in abundance ratios in molecules after low-dose or short-term labeling compared to baseline molecules before low-dose or short-term labeling, and
   (ii) by keeping the relative abundances of comparable signal intensities in the measured peaks in target molecules, to reduce the potential analytic impact of non-linearities of detector quantitation or differences in ion counting statistics; and
5) Have the potential for broad applications across biology and medicine, particularly for use of low-dose or short-term ²H₂O labeling protocols in the clinical setting.

To meet these needs, disclosed herein is a novel approach that achieves these goals by targeting a unique feature of stable isotope labeling that has not previously been used for the measurement of molecular flux rates.

### Molecular Kinetic Measurements by Quantification of Isotopologue Ratios

Quantification of molecular flux rates or kinetics by measuring abundance ratios of isotopologues in the same mass isotopomer, rather than comparisons elsewhere in the entire isotopomer envelope, constitutes a novel analytical approach for measuring flux, one which has the potential to enable greater quantitative accuracy.

As is known in the art (see, e.g., Hellerstein, M. K. and Neese, R. A. (1992) Am. J. Physiol. 263:E988-1001; and Hellerstein, M. K. and Neese, R. A. (1999) Am. J. Physiol. 276:E1146-1170), mass isotopomers are isotopic isomers of molecules that have the same elemental composition but differ in nominal mass due to different isotopic composition. Isotopologues are isotopic homologues that have the same elemental composition but differ in isotopic composition. For example, ethanol, with a molecular formula of ¹²C₂ ¹H₆ ¹⁶O₁ for the monoisotopic M0 isotopomer, has 3 possible M1 isotopologues contributing to the M1-mass isotopomer (which is nominally 1 dalton heavier than the M0 mass isotopomer); these have isotopic elemental chemical formulas of ¹³C₁ ¹²C₁ ¹H₆ ¹⁶O₁, ¹²C₂ ¹H₆ ¹⁷O₁, and ¹²O₂ ²H₁ ¹H₅ ¹⁶O₁. The ¹³C and ²H isotopologues of the M1-mass isotopomer can, in principle, be differentiated and quantified by mass spectrometry to provide information about molecular flux rates of ethanol in a biological system, after introduction of ¹³C or ²H-labeled ethanol in vivo.

The quantification of changes in relative abundance of isomeric isotopologues within mass isotopomers of a biomolecule requires an analytical measurement where the quantified analytes differ in mass by 3 to 9 thousandths of a Dalton (millidalton [mDa]), depending on which isotopologues are quantified (FIGS. 4A & 4B). The natural abundance of the stable isotope of carbon, ¹³C, is ˜1.1%, which leads to an appreciable ¹³C isotopologue contribution to the M1 isotopomer signal of carbon-containing molecules such as deoxyribonucleosides from DNA and peptides or amino acids from proteins. The ¹³C isotopologue of a typical biomolecule of these types is the most abundant molecular species in the M1- or higher mass isotopomer clusters, prior to stable isotope enrichment.

Modeled isotopomer and isotopologue mass spectra for the peptide sequence AAAEVNQEYGLDPK, which has a chemical formula of C₆₅H₁₀₁N₁₇O₂₄ and has a maximum of 34 potential deuterium incorporation sites, are shown in FIGS. 4A and 4B, respectively. The isotopomer and isotopologue abundance profiles are shown for this model peptide sequence. The monoisotopic mass isotopomer of the peptide (denoted M0) has only one isotopologue; it includes 1 molecular species with a unique isotopic elemental chemical formula and mass. The model peptide sequence for the M1 mass isotopomer cluster consists of carbon, hydrogen, nitrogen and oxygen atoms and has 4 unique elemental isotope chemical formulas or isotopologues (unique elemental formulas resulting in a nominal 1 dalton mass shift).

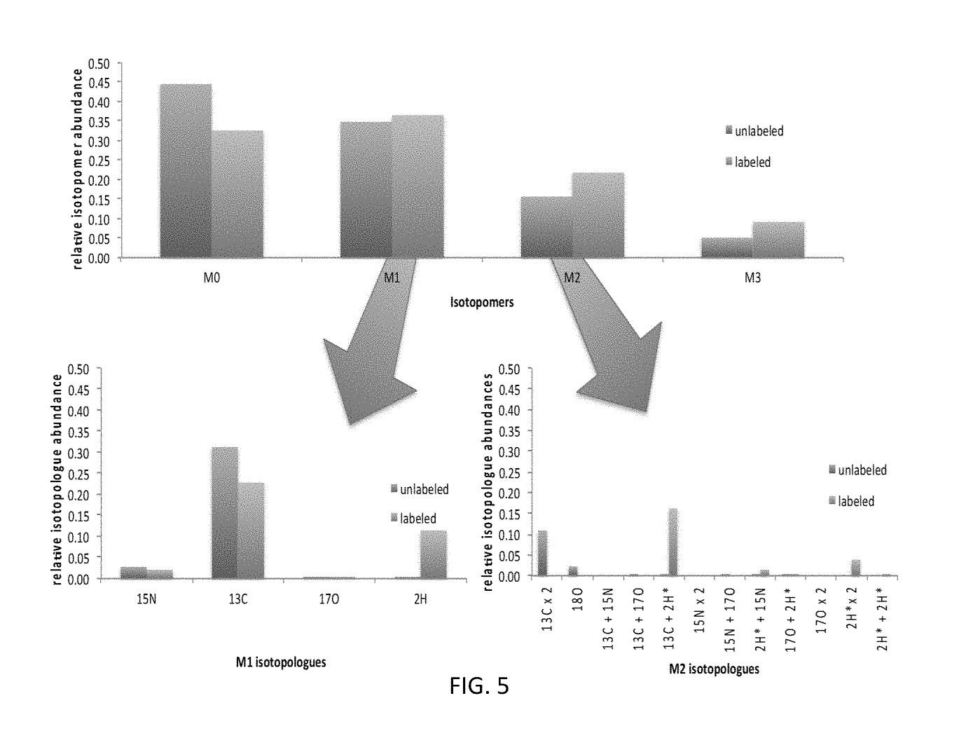

In some embodiments, e.g., wherein turnover or synthesis rates of proteins are measured by use of high resolution mass spectrometry, deuterated water (²H₂O, heavy water) may be used as the source of stable isotope label in vivo, and deuterium body water enrichment may typically be in the range of 1-2% in human subjects. For a peptide from a protein that is analyzed by mass spectrometry under these labeling conditions, with a typical deuterium enrichment in body water of 1%, the maximal change in the fractional abundance of the M0 mass isotopomer is −11.9%, or a loss of 26% of the natural abundance M0 signal intensity, as shown in FIG. 5.

Accurately measuring a loss of 26% in the M0 signal requires accurately quantifying abundances of the M1- and other mass isotopomers in the cluster for this peptide, typically M0-M3. M1 mass isotopomer, for example, will exhibit a change in fractional abundance of +1.7%. These mass isotopomers differ by ˜1 dalton each in the +1 charge state of the molecular ion. As illustrated in FIG. 5, the isotopomer cluster is therefore spread over ˜4 daltons with a wide range of relative abundances. This spread in the m/z dimension increases the likelihood of interfering analytic features or ionic contaminants relative to the more narrow mass range and ion abundances present in the isotopologue cluster within a single mass isotopomer. For a typical deuterium enrichment in body water of 1%, the maximal change in relative abundance of the ²H-isotopologue compared to the ¹³C-isotopologue in the M1 isotopomer is +11.0%, representing an increase of 3,217% over the naturally occurring isotopologue abundance of the ²H-isotopologue compared to the ¹³C-isotopologue in the M1 isotopomer (FIG. 5). This large increase in signal intensity, coincident with the very small mass difference between the ¹³C-isotopologue and the ²H-isotopologue in the M1-mass isotopomer (2.9 millidaltons) after isotope labeling, favors better analytical accuracy and precision in quantitation of relative abundances, in comparison to quantitation of mass isotopomer abundances. In addition, the likelihood of contamination by ions derived from a complex matrix (multiplexing) is obviously less over a narrow mass range such as 3 millidaltons, compared to a broad mass range such as 3 daltons.

Quantifying normalized changes in isotopologue abundance requires accurately quantifying at least two of the isotopologues in the M1-mass isotopomer cluster. It is beneficial to quantify the ¹³C and ²H isotopologue abundances, as there is only a 3 mDa mass difference (see, e.g., FIG. 4B), given that mass differences contribute to errors in measured isotope abundance in FT-ICR mass analyzers (Perry, R. H. et al. (2008) Mass. Spectrom. Rev. 27:661-699; and Scheltema, R. A. et al. (2014) Mol. Cell Proteomics 13:3698-3708).

Accordingly, theoretical accuracy for measuring the relative abundances of isotopologues within a mass isotopomer cluster may be enhanced if the relative signal intensities of the isotopologues are of comparable magnitude and the mass differences are small. To calculate the protein fractional synthesis, one can model peptide isotopologue abundances based on the precursor pool (e.g., ²H₂O) isotope enrichment and the number of stable isotope incorporation sites in each analyte (for each peptide, in this example). Based on the theoretical maximum isotopologue abundance for each peptide due to metabolic stable isotope labeling over a labeling time interval, one can then determine the fractional synthesis rate or % new protein made by comparing the isotopologue abundances measured to theoretical values for 100% and 0% new protein (Hellerstein, M. K. and Neese, R. A. (1992) Am. J. Physiol. 263:E988-1001; Hellerstein, M. K. and Neese, R. A. (1999) Am. J. Physiol. 276:E1146-1170; Hellerstein, M. K. et al. (1991) J. Clin. Invest. 87:1841-1852; Schwarz, J. M. et al. (1995) J. Clin. Invest. 96:2735-2743; Strawford, A. et al. (2004) Am. J. Physiol. Endocrinol. Metab. 286:E577-588; Neese, R. A. et al. (1995) J. Biol. Chem. 270:14452-14466; Hellerstein, M. K. et al. (1997) Am. J. Physiol. 272:E163-172; Hellerstein, M. K. et al. (1997) J. Clin. Invest. 100:1305-1309; Louie, K. B. et al. (2013) Sci. Rep. 3:1656; Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; Price, J. C. et al. (2012) Mol. Cell Proteomics 11:1801-1814; Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057).

In some embodiments, e.g., for labeling and measuring turnover rates of proteins, stable isotope labeling using 1% ¹⁵N results in an maximum ¹⁵N:¹³C isotopologue abundance ratio of 1:5 while 5% ¹⁵N pool enrichment results in a maximum ¹⁵N:¹³C isotopologue abundance ratio of approximately 1.3:1, with a mass difference of 6 mDa between these two isotopologues.

In some embodiments, cellular proliferation rates can be quantified by measuring the ¹³C and ²H isotopologue abundance ratio in the M1-mass isotopomer of deoxyribose, e.g., as isolated from genomic DNA (Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057). In an exemplary embodiment for measuring cellular proliferation, subjects drink a loading dose of 50 ml of 70% deuterated water (²H₂O) 3 times a day for 5 days, followed by 50 ml 2 times a day for 9 days. This results in a body water ²H-isotope enrichment of approximately 1-1.5%. One can then isolate nuclear DNA from human clinical samples using standard practices, liberate the sugar base from the nuclear DNA (e.g., by employing methods described in Busch, R. et al. (2007) Nat. Protoc. 2:3045-3057), and quantify the deuterium enrichment in the M1 ²H isotopologue of deoxyribose, e.g., by FT-ICR mass spectrometry. The relative isotope enrichment for deoxyribose, reflecting cellular proliferation, can be quantified by measuring the ¹³C- and ²H-M1 isotopologue abundances in the M1-mass isotopomer of deoxyribose.

By knowing the level of isotope enrichment in the subject following heavy water consumption, one can model the ¹³C and ²H isotopologue abundances as a consequence of DNA-deoxyribose synthesis during the time interval between heavy water exposure and DNA isolation. Shown in FIGS. 6A-6C are modeled isotopomer and isotopologue abundances for deoxyribose in DNA. FIG. 6A shows the modeled mass isotopomer distribution of unlabeled deoxyribose. FIG. 6B shows the modeled distribution of isotopologues in unlabeled deoxyribose. FIG. 6C the M1-distribution of isotopologue in heavy water (²H₂O) labeled deoxyribose for a body water ²H enrichment of 2%. The change in peak theoretical label % relative abundance of the ²H-isotopologue compared to the ¹³C-isotopologue of the M1-mass isotopomer is very large, as is apparent from comparing FIG. 6B to FIG. 6C.

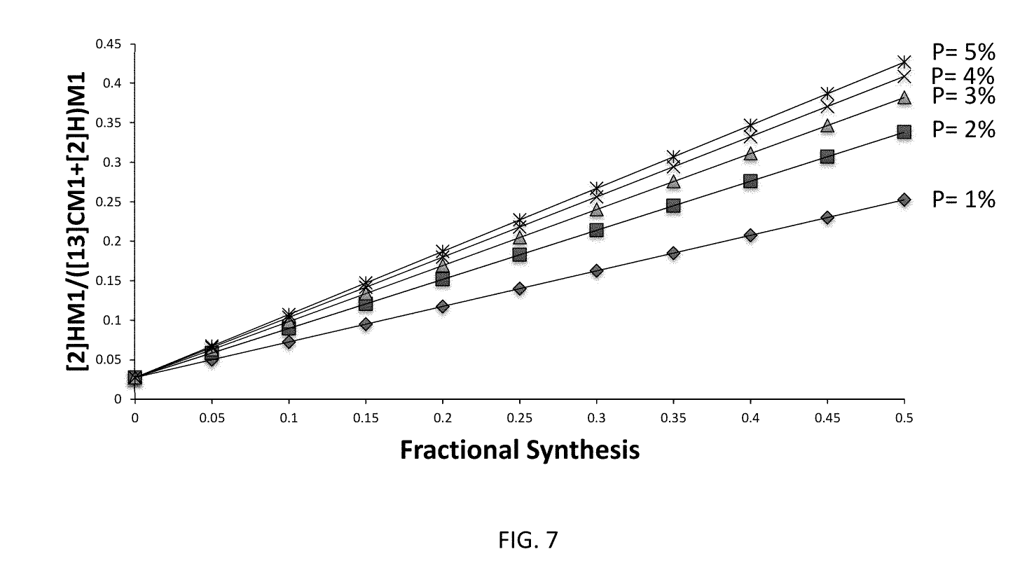

Modeling ¹³C and ²H isotopologue abundance ratios of the M1-mass isotopomer in deoxyribose at different body water ²H₂O exposures (p) demonstrates that, even at relatively low values of fractional synthesis such as 20%, the change in relative abundance of the ²H isotopologue (e.g., expressed as the abundance of ²H-isotopologue divided by the sum of the abundances of the ²H and ¹³C isotopologues) is substantial. (FIG. 7). One can quantify the ¹³C and ²H-isotopologue abundances in the M1-mass isotopomer and calculate relative abundance of the ²H-isotopologue as the ratio:

²HM1/(¹³CM1+²HM1).

This ratio reveals fractional synthesis rate (f) in a linear manner for body water enrichments (or precursor pool enrichment, abbreviated as “p” in figure) from 1-5% (FIG. 7).

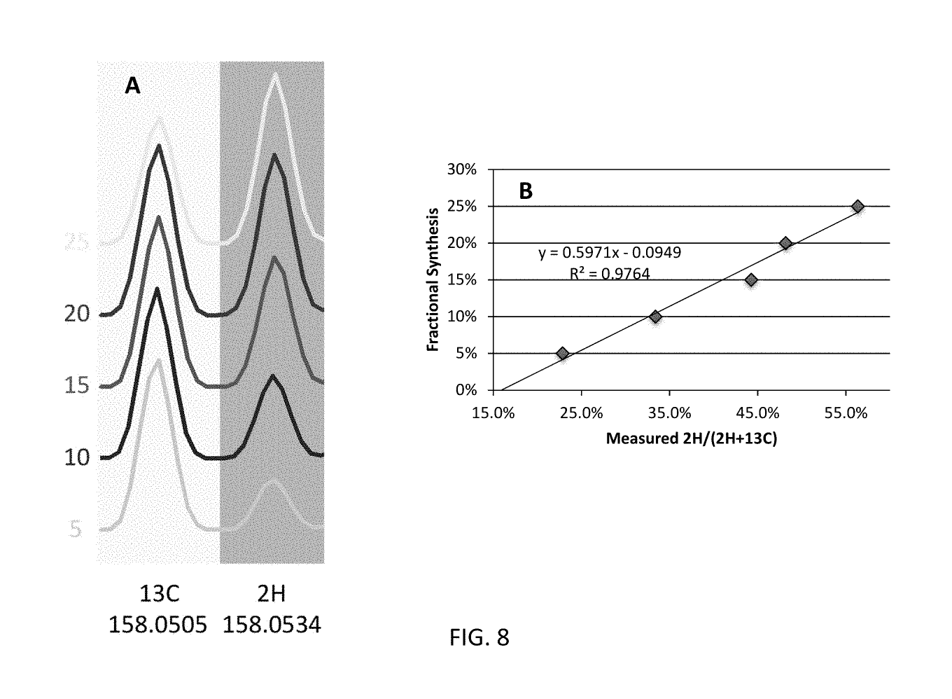

The relative abundances of isotopologues of the M1-mass isotopomer in deoxyribose isolated from DNA were quantified to calculate the fraction of newly divided cells in a mixture of labeled and unlabeled cells. Deoxyribose isolated from DNA in fully replaced (100% labeled) bone marrow cells was used as the reference values for calculation of fraction of cells newly synthesized in a mixture, as described above. The deoxyribose derived from DNA in bone marrow of rats labeled with deuterated water was isolated and was mixed with deoxyribose derived from DNA in cells from an unlabeled rat, to generate a dilution standard curve. The range 156-160 m/z was monitored by select ion monitoring (SIM) on QE-Plus with targeted ion abundance set to 3e6 (AGC) and resolution of 140K. Measurements were carried out on DNA isolated from unlabeled cells (rat liver cells) mixed with fully labeled cells (rat bone marrow cells after several weeks of heavy water intake by the animals in vivo), creating a dilution series that ranged from 0-25% labeled cells (FIG. 8A).

Isotopologue abundances were manually extracted in the 158.049-158.055 mass window and 0.17 minute time window (2.23-2.40 minute LC retention time). Isotopologue peak abundances were generated from integrating the area under the curve for an average of 9 MS scans with ˜10 points over each isotopologue peak (Table 2).

TABLE 2

| Bone Marrow labeled | Measured | Isotope abundance | |
|---|---|---|---|
| std | ²H/(²H + ¹³C) | ¹³C abundance | ²H abundance |
| 0.25 | 56.4% | 18819.6 | 24302.1 |
| 0.2 | 48.2% | 19586.6 | 18200.5 |
| 0.15 | 44.3% | 19096.6 | 15160.6 |
| 0.1 | 33.4% | 19951.8 | 10010.9 |
| 0.05 | 22.9% | 16410.1 | 4862.2 |

Measured values of abundances of mass isotopomers by GC/MS and of ²H and ¹³C-isotopologues in the M1-mass isotopomer by FT-ICR mass spectrometry are provided in Table 2 for the samples from 0% to 40% mixtures of labeled and unlabeled cells. The far right two columns show the values of the peak heights measured for the ²H and ¹³C-isotopologues in the M1-mass isotopomer.

Each spectrum was scaled to the most abundant peak; for example, in the 25% labeled DNA sample the deuterium isotopologue was more abundant and was set to 100%. A range of relative abundances of the ²H-isotopologue compared to the ¹³C-isotopologue of the M1-mass isotopomer were observed for deoxyribose (FIG. 8B).

By application of the equation:

$${( Fraction newly synthesized )}_{i} = \frac{{( observed Δ Relative {abundance}_{x} )}_{i}}{{( maximal Δ Relative {abundance}_{x} )}_{i}}$$

the fractional synthesis value can be calculated for any deoxyribose sample with relative abundance of the ²H-isotopologue compared to the ¹³C-isotopologue in the range 22.9% to 56.4% (FIG. 8B and Table 2). For example, if a sample of deoxyribose from a tissue in this labeled animal after 2 weeks of heavy water label exposure exhibited a relative abundance for the ²H-isotopologue compared to the ¹³C-isotopologue of 37.5%, calculated as [²H-isotopologue/(²H-isotopologue+¹³C-isotopologue), then using the equation in FIG. 8B (fractional synthesis=0.5971x+0.0949, where x is the relative abundance of the ²H-isotopologue, calculated as [²H-isotopologue/(²H-isotopologue+¹³C-isotopologue)]), the fraction of newly synthesized cells present is calculated to be 0.319 (31.9%).

The fractional synthesis rate (kₛ) may also be determined by applying the continuous labeling, precursor-product formula:

kₛ=[−In(1−f)]/t.

In the example described here of measuring fractional synthesis of DNA from deoxyribose labeling (see Table 2), the value of kₛ would be 0.027 per day.

It is notable that the measurement of relative abundances of isotopologues within the same mass isotopomer can reveal changes in molecular flux rates under conditions where there is a small change or no change in the relative abundance of the same mass isotopomer. This situation often occurs for ions of molecules that are of relatively high molecular weight (e.g, >1,000 daltons), where ²H-isotopologues enter and ¹³C-isotopologues leave said mass isotopomer due to molecular flux and the inflow and outflow rates roughly cancel each other out. In these instances, the larger changes in relative abundances of the ²H-isotopologue and the ¹³C-isotopologue may reveal the molecular flux.

What is claimed is:

1. A method for measuring a molecular flux rate based on analysis of isotopologue abundance within a mass isotopomer, comprising:
(a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest;
(b) obtaining from the subject a biological sample comprising the one or more stable isotope-labeled target molecules of interest;
(c) enriching or isolating the one or more stable isotope-labeled target molecules of interest from said biological sample;
(d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses;
(e) calculating a relative abundance of the first isotopologue based on the abundance of the first isotopologue and the abundance of the second isotopologue;
(f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and
(g) calculating a fraction of newly synthesized target molecules of interest based on the comparison of the relative abundance of the first isotopologue and the control relative abundance of the first isotopologue.

2. The method of claim 1, further comprising calculating a replacement rate of the target molecules of interest based on the calculated fraction of newly synthesized target molecules of interest.

3. The method of claim 1 or claim 2, further comprising obtaining from the subject at least a second biological sample comprising the one or more stable isotope-labeled target molecules of interest, wherein the first and second biological samples are obtained at different times, and wherein calculating the fraction of newly synthesized target molecules of interest comprises calculating a fraction of target molecules of interest synthesized before obtaining the first biological sample and a fraction of target molecules of interest synthesized before obtaining the second biological sample.

4. A method for measuring a molecular flux rate based on analysis of isotopologue abundance within a mass isotopomer, comprising:
(a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest;
(b) obtaining from the subject a biological sample comprising the one or more stable isotope-labeled target molecules of interest;
(c) enriching or isolating the one or more stable isotope-labeled target molecules of interest from said biological sample;
(d) performing a high resolution mass spectrometric measurement of an abundance of a first isotopologue and an abundance of a second isotopologue from said enriched or isolated one or more stable isotope-labeled target molecules of interest, wherein the first and the second isotopologues are part of the same mass isotopomer and have different masses;
(e) calculating a relative abundance of the first isotopologue based on the abundance of the first isotopologue and the abundance of the second isotopologue;
(f) comparing the relative abundance of the first isotopologue to a control relative abundance of the first isotopologue, wherein the control relative abundance is the relative abundance of the first isotopologue from the one or more target molecules of interest before or without administration of the stable isotope-labeled precursor molecule; and
(g) calculating a rate of breakdown or degradation of the target molecules of interest based on the comparison of the relative abundance of the first isotopologue and the control relative abundance of the first isotopologue.

5. The method of claim 4, further comprising calculating a replacement rate of the target molecules of interest based on the calculated rate of breakdown or degradation of the target molecules of interest.

6. The method of any one of claims 1-5, wherein the stable isotope-labeled precursor molecule is ²H₂O.

7. The method of claim 6, wherein the first isotopologue is a ²H-isotopologue, and wherein the second isotopologue is a ¹³C-isotopologue.

8. The method of any one of claims 1-5, wherein the stable isotope-labeled precursor molecule is selected from the group consisting of a ¹⁵N-labeled amino acid, a ¹⁵N-labeled polypeptide, and a ¹⁵N-labeled inorganic nitrogenous compound.

9. The method of any one of claims 1-5, wherein the stable isotope-labeled precursor molecule is selected from the group consisting of a ¹³C-labeled amino acid, a ¹³C-labeled polypeptide, a ¹³C-labeled organic metabolite, and a ¹³C-labeled inorganic carbon compound.

10. The method of any one of claims 1-5, wherein the stable isotope-labeled precursor molecule is ¹⁷O-labeled H₂O or ¹⁸O-labeled H₂O.

11. The method of any one of claims 1-10, wherein the mass isotopomer is an M1-mass isotopomer.

12. A method for measuring a molecular flux rate based on analysis of isotopologue abundance within a mass isotopomer, comprising:
(a) administering deuterated water (²H₂O) to a subject for a period of time sufficient for the deuterium of the ²H₂O to enter into a biosynthetic precursor pool and label one or more target molecules of interest to produce one or more deuterated target molecules of interest;
(b) obtaining from the subject a biological sample comprising the one or more deuterated target molecules of interest;
(c) enriching or isolating the one or more deuterated target molecules of interest from said biological sample;
(d) performing a high resolution mass spectrometric measurement of an abundance of a first isotopologue and an abundance of a second isotopologue from said enriched or isolated one or more deuterated target molecules of interest, wherein the first and the second isotopologues have different masses;
(e) calculating a relative abundance of the first isotopologue based on the abundance of the first isotopologue and the abundance of the second isotopologue;
(f) comparing the relative abundance of the first isotopologue to a control relative abundance of the first isotopologue, wherein the control relative abundance is the relative abundance of the first isotopologue from the one or more target molecules of interest before or without administration of the ²H₂O; and
(g) calculating a fraction of newly synthesized target molecules of interest based on the comparison of the relative abundance of the first isotopologue and the control relative abundance of the first isotopologue.

13. The method of claim 12, further comprising calculating a replacement rate of the target molecules of interest based on the calculated fraction of newly synthesized target molecules of interest.

14. The method of claim 12 or claim 13, further comprising obtaining from the subject at least a second biological sample comprising the one or more stable isotope-labeled target molecules of interest, wherein the first and second biological samples are obtained at different times, and wherein calculating the fraction of newly synthesized target molecules of interest comprises calculating a fraction of target molecules of interest synthesized before obtaining the first biological sample and a fraction of target molecules of interest synthesized before obtaining the second biological sample.

15. A method for measuring a molecular flux rate based on analysis of isotopologue abundance within a mass isotopomer, comprising:
(a) administering deuterated water (²H₂O) to a subject for a period of time sufficient for the deuterium of the ²H₂O to enter into a biosynthetic precursor pool and label one or more target molecules of interest to produce one or more deuterated target molecules of interest;
(b) obtaining from the subject a biological sample comprising the one or more deuterated target molecules of interest;
(c) enriching or isolating the one or more deuterated target molecules of interest from said biological sample;
(d) performing a high resolution mass spectrometric measurement of an abundance of a first isotopologue and an abundance of a second isotopologue from said enriched or isolated one or more deuterated target molecules of interest, wherein the first and the second isotopologues have different masses;
(e) calculating a relative abundance of the first isotopologue based on the abundance of the first isotopologue and the abundance of the second isotopologue;
(f) comparing the relative abundance of the first isotopologue to a control relative abundance of the first isotopologue, wherein the control relative abundance is the relative abundance of the first isotopologue from the one or more target molecules of interest before or without administration of the ²H₂O; and
(g) calculating a rate of breakdown or degradation of the target molecules of interest based on the comparison of the relative abundance of the first isotopologue and the control relative abundance of the first isotopologue.

16. The method of claim 15, further comprising calculating a replacement rate of the target molecules of interest based on the calculated rate of breakdown or degradation of the target molecules of interest.

17. The method of any one of claims 1-16, wherein the one or more target molecules of interest are peptides or amino acids isolated from one or more polypeptides.

18. The method of any one of claims 1-16, wherein the one or more target molecules of interest are deoxyribose molecules isolated from DNA.

19. The method of claim 17 or claim 18, wherein the first and the second isotopologues on which the high resolution mass spectrometric measurement is performed are derived from a fragment ion, the fragment ion being derived from the one or more stable isotope-labeled or deuterated target molecules of interest.

20. The method of any one of claims 17-19, wherein the one or more target molecules of interest are isolated from a cell.

21. The method of any one of claims 1-20, wherein the high resolution mass spectrometric measurement is performed using a high resolution mass spectrometer capable of quantifying isotopologues that differ in mass by nine or fewer millidaltons.

22. The method of claim 21, wherein the high resolution mass spectrometric measurement is performed using a high resolution mass spectrometer capable of quantifying isotopologues that differ in mass by three or fewer millidaltons.

23. The method of claim 21 or claim 22, wherein the high resolution mass spectrometer is an FT-ICR mass spectrometer.

24. The method of any one of claims 1-23, wherein the abundances of the first isotopologue and the second isotopologue are of comparable peak heights or signal intensities.

25. The method of any one of claims 1-24, wherein calculating the fraction of newly synthesized target molecules of interest, the replacement rate of the target molecules of interest, the rate of breakdown or degradation of the target molecules of interest, or any combination thereof is used as a diagnostic test.

26. The method of claim 25, wherein the diagnostic test is used in the diagnosis, management, or treatment selection of a human or veterinary patient.

27. The method of any one of claims 1-26, wherein the subject is a human.
